(19) United States
(12) Patent Application Publication
(10) Pub. No.: US 2024/0188522 A1
(43) Pub. Date: Jun. 13, 2024

(54) EGMS Rice Lines and Their F1 Hybrids

(71) Applicant: Board of Supervisors of Louisiana State University and Agricultural and Mechanical College, Baton Rouge, LA (US)

(72) Inventors: James H. Oard, Rayne, LA (US); Democrito Banay Rebong ll, Baton Rouge, LA (US)

(73) Assignee: Board of Supervisors of Louisiana State University and Agricultural and Mechanical College, Baton Rouge, LA (US)

(21) Appl. No.: 18/556,378

(22) PCT Filed: Apr. 15, 2022
(86) PCT No.: PCT/US2022/024970

§ 371 Date: Oct. 20, 2023

Related U.S. Application Data
(60) Provisional application No. 63/178,591, filed on Apr. 23, 2021.

(51) Int. Cl. A01H 5/10; A01H 6/46

(52) U.S. Cl. CPC A01H 5/10; A01H 6/4636

(57) ABSTRACT

Male-sterile EGMS rice lines, both conventional and herbicide-tolerant, and their Fi hybrids are disclosed. The EGMS lines can produce Fi hybrids with high yield, desirable agronomic traits, and good milling characteristics.

The benefit of the 23 Apr. 2021 filing date of U.S. provisional patent application Ser. No. 63/178,591 is claimed under 35 U.S.C. § 119(e) in the United States, and under applicable treaties and conventions in all countries.

### TECHNICAL FIELD

This invention pertains to male-sterile EGMS rice lines, both conventional and herbicide-tolerant, and to their F₁ hybrids.

### BACKGROUND ART

Rice is an ancient agricultural crop, and remains one of the world's principal food crops. There are two cultivated species of rice: Oryza sativa L., the Asian rice, and O. glaberrima Steud., the African rice. Oryza sativa L. constitutes virtually all of the world's cultivated rice and is the species grown in the United States. The three major rice-producing regions in the United States are the Mississippi Delta (Arkansas, Mississippi, northeast Louisiana, southeast Missouri), the Gulf Coast (southwest Louisiana, southeast Texas); and the Central Valley of California. See generally U.S. Pat. No. 6,911,589.

Rice is a semiaquatic crop that benefits from flooded soil conditions during part or all of the growing season. In the United States, rice is typically grown on flooded soil to optimize grain yields. Heavy clay soils or silt loam soils with hard pan layers about 30 cm below the surface are typical rice-producing soils, because they reduce water loss from soil percolation. Rice production in the United States can be broadly categorized as either dry-seeded or water-seeded. In the dry-seeded system, rice is sown into a well-prepared seed bed with a grain drill or by broadcasting the seed and incorporating it with a disk or harrow. Moisture for seed germination comes from irrigation or rainfall. Another method of dry-seeding is to broadcast the seed by airplane into a flooded field, and then promptly drain the water from the field. For the dry-seeded system, when the plants have reached sufficient size (four- to five-leaf stage), a shallow permanent flood of water 5 to 16 cm deep is applied to the field for the remainder of the crop season. Some rice is grown in upland production systems, without flooding.

One method of water-seeding is to soak rice seed for 12 to 36 hours to initiate germination, and then to broadcast the seed by airplane into a flooded field. The seedlings emerge through a shallow flood, or the water may be drained from the field for a short time to enhance seedling establishment. A shallow flood is then maintained until the rice approaches maturity. For both the dry-seeded and water-seeded production systems, the fields are drained when the crop is mature, and the rice is harvested 2 to 3 weeks later with large combines.

In rice breeding programs, breeders typically use the same production systems that predominate in the region. Thus, a drill-seeded breeding nursery is typically used by breeders in a region where rice is drill-seeded, and a water-seeded nursery is typically used in regions where water-seeding prevails.

Rice in the United States is classified into three primary market types by grain size, shape, and endosperm composition: long-grain, medium-grain, and short-grain. Typical U.S. long-grain cultivars cook dry and fluffy when steamed or boiled, whereas medium- and short-grain cultivars cook moist and sticky. Long-grain cultivars have been traditionally grown in the southern states and generally receive higher market prices in the U.S.

Although specific breeding objectives vary somewhat in different regions, increasing yield is a primary objective in all programs. Grain yield depends, in part, on the number of panicles per unit area, the number of fertile florets per panicle, and grain weight per floret. Increases in any or all of these components may help improve yields. Heritable variation exists for each of these components, and breeders may directly or indirectly select for any of them.

There are numerous steps in the development of any novel, desirable plant germplasm. Plant breeding begins with the analysis and definition of problems and weaknesses of the current germplasm, the establishment of program goals, and the definition of specific breeding objectives. The next step is selection (or generation) of germplasm that possesses the desired traits to meet the program goals. A goal is often to combine in a single variety an improved combination of desirable traits from two or more ancestral germplasm lines. These traits may include such things as higher seed yield, resistance to disease or insects, better stems and roots, tolerance to low temperatures, and better agronomic characteristics or grain quality.

The choice of breeding and selection methods depends on the mode of plant reproduction, the heritability of the trait(s) being improved, and the type of seed that is used commercially (e.g., F₁ hybrid, versus pure line or inbred cultivars). For highly heritable traits, a choice of superior individual plants evaluated at a single location may sometimes be effective, while for traits with low or more complex heritability, selection is often based on mean values obtained from replicated evaluations of families of related plants. Selection methods include pedigree selection, modified pedigree selection, mass selection, recurrent selection, and combinations of these methods.

The complexity of inheritance influences the choice of breeding method. Backcross breeding is used to transfer one or a few favorable genes for a highly heritable trait into a desirable cultivar. This approach has been used extensively for breeding disease-resistant cultivars. Various recurrent selection techniques are used to improve quantitatively-inherited traits controlled by numerous genes. The use of recurrent selection in self-pollinating crops depends on the ease of pollination, the frequency of successful hybrids from each pollination, and the number of hybrid offspring from each successful cross.

Promising advanced breeding lines are thoroughly tested and compared to appropriate standards in environments representative of the commercial target area(s), typically for three or more years. The best lines become candidates for new commercial cultivars; those still deficient in a few traits may be used as parents to produce new populations for further selection.

These processes, which lead ultimately to marketing and distribution of new cultivars or hybrids, typically take 8 to 12 years from the time of the first cross; they may further rely on (and be delayed by) the development of improved breeding lines as precursors. Development of new cultivars and hybrids is a time-consuming process that requires precise forward planning and efficient use of resources. There are never assurances of a successful outcome.

A particularly difficult task is the identification of individual plants that are, indeed, genetically superior. A plant's phenotype results from a complex interaction of genetics and environment. One method for identifying a genetically superior plant is to observe its performance relative to other experimental plants and to a widely grown standard cultivar raised in an identical environment. Repeated observations from multiple locations can help provide a better estimate of genetic worth.

The goal of rice breeding is to develop new, unique, and superior rice cultivars and hybrids. The breeder initially selects and crosses two or more parental lines, followed by self-pollination and selection, producing many new genetic combinations. The breeder can generate billions of different genetic combinations via crossing, selfing, and mutation breeding. The traditional breeder has no direct control of genetics at the molecular level. Therefore, two traditional breeders working independently of one another will never develop the same line, or even very similar lines, with the same traits.

Each year, the plant breeder selects germplasm to advance to the next generation. This germplasm is grown under different geographical, climatic, and soil conditions. Further selections are then made, during and at the end of the growing season. The resulting cultivars (or hybrids) and their characteristics are inherently unpredictable. This is because the traditional breeder's selection occurs in unique environments, with no control at the molecular level, and with potentially billions of different possible genetic combinations being generated. A breeder cannot predict the final resulting line, except possibly in a very gross and generic fashion. Further, the same breeder may not produce the same cultivar twice, even starting with the same parental lines, using the same selection techniques. This uncontrollable variation results in substantial effort and expenditures in developing superior new rice cultivars (or hybrids); and makes each new cultivar (or hybrid) novel and unpredictable.

The selection of superior hybrid crosses is somewhat different. Hybrid seed is typically produced by manual crosses between selected male-fertile parents, or by using genetic male sterility systems. These hybrids are typically selected for single gene traits that unambiguously indicate that a plant is indeed an F₁ hybrid with inherited traits from both presumptive parents, particularly the male parent (since rice normally self-fertilizes). Such traits might include, for example, a semi-dwarf plant type, pubescence, awns, or apiculus color. Additional data on parental lines, as well as the phenotype of the hybrid, influence the breeder's decision whether to continue with a particular hybrid cross or an analogous cross, using related parental lines.

Pedigree breeding and recurrent selection breeding methods are sometimes used to develop cultivars from breeding populations. These breeding methods combine desirable traits from two or more cultivars or other germplasm sources into breeding pools from which cultivars are developed by selfing and selection of desired phenotypes. The new cultivars are evaluated to determine commercial potential.

Pedigree breeding is often used to improve self-pollinating crops. Two parents possessing favorable, complementary traits are crossed to produce F₁ plants. An F₂ population is produced by selfing one or more F₁s. Selection of the superior individual plants may begin in the F₂ (or later) generation. Then, beginning in the F₃ (or other subsequent) generation, individual plants are selected. Replicated testing of panicle rows from the selected plants can begin in the F₄ (or other subsequent) generation, both to fix the desired traits and to improve the effectiveness of selection for traits that have low heritability. At an advanced stage of inbreeding (e.g., F₆ or F₇), the best lines or mixtures of phenotypically-similar lines are tested for potential release as new cultivars.

Mass and recurrent selection methods can also be used to improve populations of either self- or cross-pollinating crops. A genetically variable population of heterozygous individuals is either identified or created by intercrossing several different parents. The best offspring plants are selected based on individual superiority, outstanding progeny, or excellent combining ability. The selected plants are intercrossed to produce a new population in which further cycles of selection are continued.

Backcross breeding is often used to transfer genes for a simply inherited, highly heritable trait into a desirable homozygous cultivar or inbred line, which is the recurrent parent. The source of the trait to be transferred is called the donor parent. The resulting plant should ideally have the attributes of the recurrent parent (e.g., cultivar) and the desired new trait transferred from the donor parent. After the initial cross, individuals possessing the desired donor phenotype (e.g., disease resistance, insect resistance, herbicide tolerance) are selected and repeatedly crossed (backcrossed) to the recurrent parent.

The single-seed descent procedure in the strict sense refers to planting a segregating population, harvesting a sample of one seed per plant, and using the one-seed sample to plant the next generation. When the population has been advanced from the F₂ generation to the desired level of inbreeding, the several plants from which the lines are derived will each trace to different F₂ individuals. The number of plants in a population declines each generation, due to failure of some seeds to germinate or the failure of some plants to produce at least one seed. As a result, not all of the F₂ plants originally sampled in the population will be represented by progeny in subsequent generations.

In a multiple-seed procedure, the breeder harvests one or more seeds from each plant in a population and threshes them together to form a bulk. Part of the bulk is used to plant the next generation and part is held in reserve. The procedure has been referred to as modified single-seed descent or the pod-bulk technique. The multiple-seed procedure has been used to save labor at harvest. It is considerably faster to thresh panicles by machine than to remove one seed from each by hand as in the single-seed procedure. The multiple-seed procedure also makes it possible to plant the same number of seeds from a population for each generation of inbreeding. Enough seeds are harvested to compensate for plants that did not germinate or produce seed.

Other common and less-common breeding methods are known and used in the art. See, e.g., R. W. Allard, Principles of Plant Breeding (John Wiley and Sons, Inc., New York, New York, 1967); N. W. Simmonds, Principles of Crop Improvement (Longman, London, 1979); J. Sneep et al., Plant Breeding Perspectives (Pudoc, Wageningen, 1979); and W. R. Fehr, Principles of Cultivar Development: Theory and Technique (Macmillan Pub., New York, New York, 1987).

Proper testing should detect any major faults and establish the level of superiority or improvement over current cultivars or hybrids. In addition to showing superior performance, there must be a demand for a new cultivar or hybrid; i.e., the new cultivar or hybrid should either be compatible with industry standards, or it should create a new market. The introduction of a new cultivar or hybrid may incur additional costs to the seed producer, the grower, processor, and consumer for such things as special advertising and marketing, altered seed and commercial production practices, and new product utilization. The testing that precedes the release of a new cultivar or hybrid should take into account research and development costs, in addition to technical superiority of the final cultivar or hybrid.

U.S. Pat. Nos. 7,019,196 and 9,090,904 disclose herbicide-tolerant rice plants that are resistant to (or tolerant of) certain herbicides that normally inhibit the growth of rice plants, namely certain imidazolinone and sulfonylurea herbicides. With herbicide-tolerant rice plants, rice growers can control weeds that previously were difficult to control in rice fields, including “red rice.” “Red rice” is a weedy relative of cultivated rice, which had previously been difficult to control because it actually belongs to the same genus (Oryza), and sometimes even the same species (O. sativa) as cultivated rice. Only in recent years, when herbicide tolerant rice cultivars first became available, did it become possible to control red rice with herbicides in fields where cultivated rice was growing contemporaneously.

Unfortunately, growers using this system have observed both the outcrossing of herbicide tolerance from cultivated herbicide-tolerant rice lines into red rice, and also the selective pressure favoring evolution of herbicide-tolerant weeds generally-weeds that include both red rice and other weeds. There is an unfilled need for new herbicide-tolerant rice cultivars and herbicide-tolerant rice hybrids—that is, rice plants that not only express a desired herbicide-tolerant phenotype, but that also possess other agronomically desirable characteristics. Additional herbicide-tolerant cultivars and hybrids will provide rice growers greater flexibility in planting and managing crops. There is in particular an unfilled need for new herbicide-tolerant rice cultivars and rice hybrids that are tolerant to different categories of herbicide, so that red rice and other weeds that may have acquired resistance to imidazolinones and sulfonylureas can still be controlled using an alternative herbicide chemistry. The ability to rotate rice fields between herbicide-tolerant crops that are tolerant to different families of herbicides would be of great benefit both to growers and to consumers.

Published patent applications US 2016/0264990, WO 2018/236802, and WO 2020/219346 disclose rice plants that are tolerant to herbicides that inhibit acetyl-Coenzyme A carboxylase activity at levels of herbicide that would normally inhibit the growth of a rice plant.

### DISCLOSURE OF THE INVENTION

We have discovered four novel environment-sensitive genic male sterility (EGMS) rice lines that are useful, for example, as parental lines in breeding F₁ rice hybrids. Two of the novel lines are herbicide-tolerant, exhibiting tolerance to herbicides that inhibit acetyl-Coenzyme A carboxylase activity. The other two novel lines are conventional rice lines, meaning that they are not herbicide-tolerant. The lines produced hybrids in combination with specific pollinators that were comparable to commercial hybrid check lines. Overall results indicated that the selected EGMS lines can produce hybrids with high yield, desirable agronomic traits, and good milling characteristics.

This invention also pertains to methods for producing a hybrid or a new variety by crossing an EGMS line with another rice line, one or more times. Such methods of using the novel rice lines are also aspects of this invention, including backcrossing, hybrid production, crosses to populations, and other breeding methods. Hybrid plants produced using the one of the EGMS lines as a parent are also within the scope of this invention, particularly including but not limited to F₁ hybrids.

In another embodiment, this invention allows for single-gene converted plants of the EGMS lines. The single transferred gene may be a dominant or recessive allele. Preferably, the single transferred gene confers a trait such as resistance to insects; resistance to one or more bacterial, fungal, or viral diseases; male fertility or sterility; enhanced nutritional quality; enhanced processing qualities; or an additional source of herbicide resistance. The single gene may be a naturally occurring rice gene or a transgene introduced through genetic engineering techniques known in the art. The single gene also may be introduced through traditional backcrossing techniques or genetic transformation techniques known in the art.

In another embodiment, this invention provides regenerable cells for use in tissue culture of the EGMS rice lines or their hybrids. The tissue culture may allow for regeneration of plants having physiological and morphological characteristics of the EGMS line (or hybrid) and of regenerating plants having substantially the same genotype as the original EGMS line (or hybrid). Tissue culture techniques for rice are known in the art. The regenerable cells in tissue culture may be derived from sources such as embryos, protoplasts, meristematic cells, callus, pollen, leaves, anthers, root tips, flowers, seeds, panicles, or stems. In addition, the invention provides rice plants regenerated from such tissue cultures.

In another embodiment, the present invention provides a method for producing rice; the method comprises germinating, or planting and germinating, a rice seed, and growing therefrom a plant to produce a phenotype of whole plant tolerance to a herbicide, wherein said rice belongs to any of (a) one of the herbicide-tolerant EGMS lines, or (b) an F₁ or later-generation hybrid, derivative, or progeny of one of these lines that expresses the environment-sensitive genic male sterility characteristics of the EGMS line; and optionally that also expresses the herbicide resistance characteristics of the EGMS line. In another embodiment, the present invention provides a method for producing rice; the method comprises growing rice plant(s) in the presence of a herbicide to which it is tolerant, and optionally comprises selecting rice plant(s) based on tolerance to the herbicide, wherein said rice belongs to any of (a) one of the herbicide-tolerant EGMS lines or (b) a hybrid, derivative, or progeny of one of the herbicide-tolerant EGMS lines that expresses the herbicide resistance characteristics of the original line. These methods can optionally include a step of harvesting rice seed from the rice plant(s). In some embodiments, the rice plant(s) exhibit one or more of: tolerance to a cycloxydim herbicide applied at a 100 g AI/ha rate, or at a 200 g AI/ha rate; tolerance to a haloxyfop herbicide applied at a 100 g AI/ha rate, or at a 200 g AI/ha rate; tolerance to a quizalofop herbicide; or tolerance to a tepraloxdyim herbicide.

In another embodiment, the present invention provides a method for producing rice by breeding one of the herbicide-tolerant EGMS lines, or a hybrid, derivative, or progeny plant thereof that comprises the herbicide resistance phenotype of one of the herbicide-tolerant EGMS lines with another rice plant to produce a new plant, the new plant having the ACCase herbicide resistance phenotype of one of the herbicide-tolerant EGMS lines. Such a breeding process can involve step(s) of out-crossing, back-crossing, and/or self-crossing, whether by pollination, fusion of rice cell nuclei, or any other method known in the art. In some embodiments, the breeding process can comprise step(s) involving chromosome doubling, embryo rescue, plantlet regeneration, or other techniques known in the art. In some embodiments of the method, said breeding includes incorporating an additional or modified gene in the new plant (or its seed) that was, respectively, absent from or unmodified in the original plant or in its hybrid, derivative, or progeny plant. In some embodiments, the additional or modified gene can be expressed by the new plant to produce a phenotype of: herbicide tolerance (e.g., using an herbicide-resistant ACCase, AHAS, CYP450, EPSPS, GAT, GOX, HPPD, HST, PAT/bar, or PPX gene), pest or disease resistance (e.g., resistance toward one or more insect, nematode, fungus, bacteria, or virus), stress resistance (e.g., resistance toward water stress, salinity, or heat stress), or altered biomolecule synthesis (e.g., increased or decreased biosynthesis of one or more fatty acid, carbohydrate, polypeptide, or secondary metabolite), or other phenotypic trait gene known in the art. In some embodiments, the additional or modified gene is non-transgenic; in some embodiments, it is a transgene.

In another embodiment, the invention provides a method for preparing a polynucleotide encoding the herbicide tolerant ACCase polypeptide of a plant of one of the herbicide-tolerant EGMS lines, or of progeny thereof, by obtaining biological material from the plant or a seed thereof, and isolating from the material a nucleic acid, or a copy thereof, that has the coding sequence for the herbicide-tolerant ACCase polypeptide. The isolation may involve one or more of, e.g., genomic DNA cloning, PCR amplification, reverse transcription, polynucleotide sequencing, or any other technique known in the art as useful for such isolation. The copy of the nucleic acid can be, e.g., a cDNA prepared from an mRNA of the plant biomaterial, a PCR amplified copy of genomic DNA of the plant biomaterial, or a de novo synthesized nucleic acid, prepared using sequence information obtained from nucleic acid of the plant biomaterial.

In another embodiment, the invention provides a method for producing a herbicide-tolerant plant comprising transforming, into a plant tissue or cell, e.g., a rice tissue or cell, the herbicide-tolerantACCase-encoding polynucleotide isolated from one of the herbicide-tolerant EGMS lines, or of progeny thereof, or a copy of said polynucleotide. Any transformation techniques known in the art as useful therefor can be employed. The resulting transformed tissue or cell can be regenerated to form plantlet(s) that can be grown to produce mature plant(s).

In another embodiment, the invention provides a method for preparing, in rice or another plant species, a gene-edited version of an ACCase polynucleotide encoding the herbicide-tolerant ACCase polypeptide of a plant of one of the herbicide-tolerant EGMS lines or of its homolog in another plant species, e.g., of another Poaceae species. This method comprises using the polynucleotide or amino sequence of an ACCase nucleic acid or polypeptide of a plant of one of the herbicide-tolerant EGMS lines. Oligonucleotide-directed mutagenesis, mismatch-repair oligonucleotide treatment, CRISPR-Cas, or any other gene-editing technology known in the art can be used to produce a herbicide-tolerance substitution identical to (or homologous to) that present in the herbicide-tolerant plastidic ACCase of a plant of one of the herbicide-tolerant EGMS lines i.e. I1781(Am)L, based on sequence data obtained from a sample of biomaterial of one of the herbicide-tolerant EGMS lines or of a progeny thereof, e.g., seed, leaf, or other biomaterial.

In another embodiment, the present invention provides a method for controlling weeds in the vicinity of rice. The method comprises contacting the rice, and preferably weeds in the vicinity of the rice, with a herbicide, wherein said rice belongs to any of (a) one of the herbicide-tolerant EGMS lines or (b) a hybrid, derivative, or progeny of one of the herbicide-tolerant EGMS lines that expresses the herbicide resistance characteristics of one of the herbicide-tolerant EGMS lines.

In some embodiments, the herbicide is an acetyl CoA carboxylase-inhibiting herbicide. Acetyl-CoA carboxylase (“ACCase”) is a biotin-dependent enzyme that catalyzes the irreversible carboxylation of acetyl-CoA to produce malonyl-CoA. Several ACCase-inhibiting herbicides are known in the art, such as one or more of the aryloxyphenoxy (FOP) herbicides, one or more the cyclohexanedione (DIM) herbicides, or combinations thereof.

In one embodiment, the rice is a rice plant, and said contacting comprises applying the herbicide in the vicinity of the rice plant.

In another embodiment, the herbicide is applied to weeds in the vicinity of the rice plant.

In still further embodiments, the rice is rice seed, and said contacting comprises applying the herbicide to the rice seed.

In some embodiments, the present invention provides a method for treating rice. The method comprises contacting the rice with an agronomically acceptable composition, wherein said rice belongs to any of (a) one of the herbicide-tolerant EGMS lines or (b) a hybrid, derivative, or progeny of one of the herbicide-tolerant EGMS lines that expresses the ACCase herbicide resistance characteristics of one of the herbicide-tolerant EGMS lines.

In one embodiment, the agronomically acceptable composition comprises at least one agronomically acceptable active ingredient.

In another embodiment, the agronomically acceptable active ingredient is selected from the group consisting of fungicides, insecticides, antibiotics, stress tolerance-enhancing compounds, growth promoters, herbicides, molluscicides, rodenticides, animal repellants, and combinations thereof.

In some embodiments, the present invention provides a progeny rice line or variety obtainable from one of the four novel EGMS rice lines, a representative sample of seeds of each of the four EGMS lines having been deposited (as described in greater detail below) under the Budapest Treaty.

In other embodiments, the present invention provides a method for controlling weeds in a field, said method comprising: growing a herbicide-tolerant plant according to the present invention in a field; and contacting said plant and weeds in the field with an effective amount of an ACCase-inhibiting herbicide to which the plant is tolerant, thereby controlling weeds in the field without adversely affecting the cultivated rice plant.

In some embodiments, improved rice plants and rice lines having tolerance to at least one ACCase-inhibitor herbicide are provided. In some embodiments, the ACCase-inhibitor herbicide is an aryloxyphenoxypropionate (FOP) herbicide, a cyclohexanedione (DIM) herbicide, a phenylpyrazoline (DEN) herbicides, an agronomically acceptable salt or ester of one of these, or a combination thereof. Examples of such herbicides include: DIMs, e.g., cycloxydim, sethoxydim, clethodim, or tepraloxydim; FOPs, e.g., clodinafop, diclofop, fluazifop, haloxyfop, or quizalofop; and DENs, e.g., pinoxaden, Preferred esters of quizalofop or quizalofop-P include the ethyl and tefuryl esters; and preferred esters of haloxyfop or haloxyfop-P include the methyl and etotyl esters.

The rice plants and rice lines of the present invention also provide for improved systems and methods for controlling weeds using at least one ACCase-inhibitor herbicide.

### Definitions

The following definitions apply throughout the specification and claims, unless context clearly indicates otherwise:

“Days to 50% heading.” Average number of days from seeding to the day when 50% of all panicles are exerted at least partially through the leaf sheath. A measure of maturity.

“Grain Yield.” Grain yield is measured in pounds per acre, at 12.0% moisture. Grain yield depends on a number of factors, including the number of panicles per unit area, the number of fertile florets per panicle, and grain weight per floret.

“Lodging Percent.” Lodging is a subjectively measured rating, and is the percentage of plant stems leaning or fallen completely to the ground before harvest.

“Grain Length (L).” Length of a rice grain, or average length, measured in millimeters.

“Grain Width (W).” Width of a rice grain, or average width, measured in millimeters.

“Length/Width (L/W) Ratio.” This ratio is determined by dividing the average length (L) by the average width (W).

“1000 Grain Wt.” The weight of 1000 rice grains, measured in grams.

“Harvest Moisture.” The percentage moisture in the grain when harvested.

“Plant Height.” Plant height in centimeters, measured from soil surface to the tip of the extended panicle at harvest.

“Apparent Amylose Percent.” The percentage of the endosperm starch of milled rice that is amylose. The apparent amylose percent is an important grain characteristic that affects cooking behavior. Standard long grains contain 20 to 23 percent amylose. Rexmont-type long grains contain 24 to 25 percent amylose. Short and medium grains contain 13 to 19 percent amylose. Waxy rice contains zero percent amylose. Amylose values, like most characteristics of rice, depend on the environment. “Apparent” refers to the procedure for determining amylose, which may also involve measuring some long chain amylopectin molecules that bind to some of the amylose molecules. These amylopectin molecules actually act similar to amylose in determining the relative hard or soft cooking characteristics.

“Alkali Spreading Value.” An index that measures the extent of disintegration of the milled rice kernel when in contact with dilute alkali solution. It is an indicator of gelatinization temperature. Standard long grains have a 3 to 5 Alkali Spreading Value (intermediate gelatinization temperature).

“Peak Viscosity.” The maximum viscosity attained during heating when a standardized, instrument-specific protocol is applied to a defined rice flour-water slurry.

“Trough Viscosity.” The minimum viscosity after the peak, normally occurring when the sample starts to cool.

“Final Viscosity.” Viscosity at the end of the test or cold paste.

“Breakdown.” The peak viscosity minus the hot paste viscosity.

“Setback.” Setback 1 is the final viscosity minus the trough viscosity. Setback 2 is the final viscosity minus the peak viscosity.

“RVA Viscosity.” Viscosity, as measured by a Rapid Visco Analyzer, a widely used laboratory instrument to examine the paste viscosity or thickening ability of milled rice during the cooking process.

“Hot Paste Viscosity.” Viscosity measure of rice flour/water slurry after being heated to 95° C. Lower values indicate softer and stickier cooking types of rice.

“Cool Paste Viscosity.” Viscosity measure of rice flour/water slurry after being heated to 95° C. and uniformly cooled to 50° C. Values less than 200 indicate softer cooking types of rice.

“Allele.” An allele is any of one or more alternate forms of the same gene. In a diploid cell or organism such as rice, the two alleles of a given gene occupy corresponding loci on a pair of homologous chromosomes.

“Backcrossing.” Backcrossing is a process in which a breeder repeatedly crosses hybrid progeny back to one of the parents, for example, crossing a first generation hybrid F₁ with one of the parental genotypes of the F₁ hybrid, and then crossing a second generation hybrid F₂ with the same parental genotype, and so forth.

“Essentially all the physiological and morphological characteristics.” A plant having “essentially all the physiological and morphological characteristics” of a specified plant refers to a plant having the same general physiological and morphological characteristics, except for those characteristics that are derived from a particular converted gene.

“Quantitative Trait Loci (QTL).” Quantitative trait loci (QTL) refer to genetic loci that to some degree control numerically measurable traits, generally traits that are continuously distributed.

“Regeneration.” Regeneration refers to the development of a plant from tissue culture.

“Single Gene Converted (Conversion).” Single gene converted (conversion) includes plants developed by backcrossing, wherein essentially all of the desired morphological and physiological characteristics of a parental variety are recovered, while also retaining a single gene that is transferred into the plants via crossing and backcrossing. The term can also refer to the introduction of a single gene through genetic engineering techniques known in the art.

No distinction is intended between “herbicide tolerance” and “herbicide resistance” (or similar terms). The two terms are used interchangeably, as are related terms such as “herbicide tolerant” and “herbicide resistant.”

### MODES FOR CARRYING OUT THE INVENTION

Two of the Budapest Treaty-deposited EGMS lines, 16HT195 and 16HT695, are tolerant to ACCase herbicides. For example, they are tolerant to Quizalofop-P-ethyl, the generic name for ethyl (R)-2-[4-(6-chloroquinoxalin-2-yl) oxy)phenoxy]propionate, which is sold under the tradename Provisia™ herbicide. Two of the Budapest Treaty-deposited EGMS lines, 16HT489 and 16HT498, are “conventional,” meaning they are not herbicide-tolerant.

The herbicide tolerance for 16HT195 is derived from the line BASF 1-15. The herbicide tolerance for 16HT695 is derived from the line BASF 1-14. BASF 1-15 and BASF 1-14 are both proprietary rice line, not publicly released, that carry resistance to ACCase herbicides. BASF 1-14 and BASF 1-15 both comprises a gene with a mutagenized rice nucleic acid encoding a rice plastidic ACCase enzyme. As a result of mutagenesis, the rice plastidic ACCase gene in BASF 1-14 and BASF 1-15 both have a leucine amino acid residue substituted for an isoleucine amino acid residue at position 1792 in the rice ACCase amino acid sequence, a position that corresponds to amino acid position 1781 of the plastidic ACCase of Alopecurus myosuroides. (The A. myosuroides (blackgrass) plastidic ACCase amino acid sequence is generally used in the art to define the standard numbering of amino acid positions for plastidic ACCases in grasses.) In Poaceae (grasses) the plastidic ACCase a single-chain protein. The mutagenized plastidic ACCase is responsible for herbicide tolerance. The substitution in the mutant rice allele can be described with the nomenclature I1781(Am)L, referring to the corresponding position in A. myosuroides. Alternatively, the substitution could also be described with the nomenclature I1792(Os)L, referring to the position in of the substitution in Oryza sativa.

The novel herbicide-tolerant plants comprise, as a result of direct inheritance and selection at each generation, the herbicide-tolerance trait from the rice line ‘OsHPHI2,’ a representative sample of seed of which has been deposited under the Budapest Treaty with the American Type Culture Collection under Accession No. PTA-10267. The production of rice line ‘OsHPHI2’ is described in published international patent application WO2011/028832, the entire disclosure of which is hereby incorporated by reference. A nuclear gene encodes the plastidic (chloroplast) ACCase. The rice plastidic ACCase is 2327 AA long (the blackgrass sequence is 2320 AA long). WO2011/028832 discloses the sequence; the wild-type sequence is shown therein as sequence number three. The I1781(Am)L mutation in the rice plastidic ACCase provides tolerance to a range of ACCase-inhibiting herbicides of varying chemical classes—for example, quizalofop, cycloxydim, and others described below.

Both the herbicide-tolerant and the conventional EGMS lines are adapted for production throughout the southern United States rice production area, including Louisiana, Texas, Mississippi, Arkansas, and Missouri. These lines and their hybrids will also be suited for production in rice production areas in other countries. These lines may be crossed with lines adapted for particular production areas, and the hybrids or offspring selected for suitability for particular production areas.

Our motivation was to develop two-line hybrids with high yields; some of which would carry resistance to the herbicide Quizalofop-P-Ethyl (QPE) to control weedy red rice, and help prolong Clearfield® and Provisia® weed control technologies. A first objective of this research was to develop and characterize QPE-resistant, environment-sensitive genic male sterility (EGMS) lines adapted to Louisiana field conditions. Five promising EGMS lines with desired agronomic traits were selected and evaluated from 2017-2020. A second objective was to evaluate field performance of QPE-resistant experimental hybrids derived from crosses of the selected EGMS lines. Three QPE-resistant hybrids with high yields comparable to commercial hybrids were identified in 2019 and 2020 trials. A third objective of this research was to develop and optimize seed production methods for parental lines and hybrids under Louisiana field conditions. Crossing blocks with three rows of female and male plants planted at the same time allowed sufficient seed production for small-plot yield trials. Our research helped optimize plot layout, planting date, seeding rate, and timing of GA₃ plant growth regulators for enhanced parental and hybrid seed production for two-line hybrid breeding.

Red rice is one of the most important weed species. Its weedy traits include rapid early growth, taller canopy than cultivated rice, tendency to lodge, high tillering capacity, spreading growth habit, high consumption of fertilizer, tolerance to shade, asynchronous maturation of grains, seed dehiscence, seed dormancy and a red pericarp. Red rice has been spread largely by planting contaminated commercial seeds and movement of equipment from infested fields to non-infested areas. With its red pericarp, red rice reduces the market value of commercial rice.

Three-line hybrids can have a 15 to 20 percent higher yield than inbred cultivars. A three-line hybrid rice system involves three parents: the CMS (A) line, a maintainer (B) line, and a restorer (R) line. A CMS line is multiplied or reproduced by crossing it with a maintainer line—the two lines are morphologically similar, except that the A-line is male-sterile and the B-line is male-fertile. The restorer or R-line possesses dominant fertility-restoring genes that, when crossed with the CMS (A) line, restores fertility in the derived F₁ hybrid.

While the three-line hybrids have been successful, further research has led to a successful two-line hybrid rice system. The two-line system employs an environment-sensitive genic male sterility (EGMS) trait, as an alternative to the CMS used in a three-line system. Not only is a two-line system easier to work with, two-line hybrids can also have a 5 to 10 percent yield advantage over even three-line hybrids.

Acreage planted to hybrids in Louisiana was practically non-existent only a few years ago, but reached 33% in 2020.

The two-line hybrid rice system used in one embodiment is based on two types of EGMS lines: photosensitive (PGMS) and thermosensitive (TGMS) genetic male sterile lines. The PGMS and TGMS traits themselves were developed in China. Male sterility is controlled by one or two pairs of recessive nuclear genes. See, e.g.: C. De Guzman et al., “Genetic Analysis of Photoperiod/Thermosensitive Male Sterility in Rice under US Environments,” Crop Science, vol. 57, pp. 1957-1965 (2017); C. De Guzman et al., “Genetic Analysis of Environment-Sensitive Genic Male Sterile Rice under US Environments,” Euphytica, vol. 215:39 (2019); A. Abbas et al., “Exploiting Genic Male Sterility in Rice: From Molecular Dissection to Breeding Applications,” Frontiers in Plant Science, vol. 12, article 629314 (2021); H. Zhou et al., “Photoperiod- and thermo-sensitive genic male sterility in rice are caused by a point mutation in a novel noncoding RNA that produces a small RNA,” Cell Research, vol. 22, pp. 649-660 (2012); S. Virmani, Heterosis and Hybrid Rice Breeding, Monographs on Theoretical and Applied Genetics, vol. 22 (R. Frankel et al., eds. 1994); and S. Virmani et al., Two-Line Hybrid Rice Breeding Manual (International Rice Research Institute 2003).

See also J. Camacho et al., “Development and Evaluation of Hybrid Rice Resistant to the ACCase Inhibitor Quizalofop-P-Ethyl,” (Abstract and Slides, Rice Technical Working Group, 21 Feb. 2018, Long Beach, California); D. Rebong et al., “Development of Candidate Provisia® Hybrids for the Louisiana Rice Industry (Abstract and Slides, Rice Technical Working Group, 26 Feb. 2020, Perdido Beach Resort, Alabama); and “Theses and Dissertations, 2020-2021,” CSA News, pp. 47-56, particularly p. 48 (February 2022).

Using these traits, we have bred, characterized, and evaluated selected EGMS lines as a step in developing QPE-resistant hybrids.

Purity and quality of EGMS lines are important for commercial production. For every 1% decrease in purity of hybrid seeds, there is an estimated 100 kg/ha yield loss in production. Hybrid rice seed production methods require care to minimize costs and maximize returns. Among the factors that affect hybrid seed production are synchronization of flowering, isolation method, row ratio, rogueing, timing, and rate of gibberellic acid (GA) treatment, and supplementary pollination. Our research has found preferred conditions to enhance seed production for parents and hybrids in field and greenhouse environments.

We have developed and characterized QPE-resistant, EGMS lines adapted to (at least) Louisiana field conditions; evaluated field performance of QPE-resistant experimental hybrids developed using the QPE-resistant, EGMS lines; and we have developed preferred seed production methods for parents and hybrids under Louisiana field and greenhouse conditions.

### Development of Characterization of QPE-Resistant EGMS Lines Adapted to Louisiana Field Conditions

The PGMS lines are generally sterile with daylight longer than ˜13 h, and are partially fertile with daylight less than ˜13 h. The TGMS lines are generally sterile at higher temperatures (day ≳30° C., night ≳24° C.), and partially fertile at lower temperatures (day 24° C., night ≲16° C.).

A two-line hybrid system has several advantages over a three-line system. A two-line system does not need for a fertile (maintainer) line for seed multiplication of the female parent, which simplifies hybrid seed production. Another is that any fertile line can be used as the male parent, while the three-line system requires that male parents contain the fertility restorer (Rf) gene. Thus there is more flexibility in developing elite commercial two-line hybrids than three-line hybrids. The extent of heterosis in the two-line system can be higher because the negative effects of a sterility-inducing cytoplasm are avoided.

An EGMS system can be incorporated into almost any genetic background, providing greater genetic and cytoplasmic diversity in male sterile lines, and reducing the risk of genetic vulnerability in the hybrids. A two-line system is ideal for developing hybrids with higher heterosis viz., O. indica/O. japonica crosses.

However, EGMS carries some disadvantages. The EGMS system relies on environmental conditions, which may change from location to location. EGMS seed propagation and hybrid seed production do best in locations with consistent weather conditions during the reproductive stage. Because the critical temperature/photoperiod of EGMS lines can shift after a few generations, re-isolation of EGMS lines with the desired temperature/photoperiod may be needed periodically. The genetic background is not a neutral player in an EGMS system, as the general genetic background can itself influence the critical environmental conditions needed for fertility restoration, which can introduce creating challenges when breeding EGMS lines.

### Materials and Methods

EGMS line 2009S was obtained from the Guangxi Academy of Agricultural Sciences, China. The EGMS trait in 2009S is single-gene recessive, with typical thermosensitive behavior and little or no response to changes in photoperiod. Higher pollen fertility results when the plant experiences lower temperatures for at least ten days while the flag leaf is not yet fully exposed; pollen sterility occurs at higher temperatures (28-30° C.).

Quizalofop-P-Ethyl (QPE) herbicide resistance under field conditions is governed by a single dominant gene that segregates in a Mendelian fashion, with no observed maternal effects. For initial development of QPE-resistant EGMS lines, sister lines of 2009S were crossed with different QPE-resistant O. indica lines obtained from BASF, followed by subsequent topcrosses to Louisiana cultivars Cocodrie, Cheniere, Catahoula, and Mermentau. These breeding efforts in 2017 resulted in the development of 238 QPE-resistant EGMS lines in the F_{3:4}, F_{4:5}, and F_{5:6} generations.

Of these 238 QPE-resistant EGMS lines, 100 EGMS lines were obtained from single crosses between the 2009S sister lines and QPE-resistant indica lines (BASF 1-8, 1-12, 1-14, 1-15 and 2-31). The other 138 EGMS lines were developed from topcrosses between the F₁ single crosses, and the locally-adapted, long-grain tropical O. japonica varieties Cocodrie, Cheniere, Catahoula, and Mermentau (i.e., three-way crosses).

Evaluation, selection, and advancement of EGMS lines were based on a combination of target traits and single nucleotide polymorphism (SNP) marker profiles. The EGMS lines were evaluated for pollen sterility, days to emergence, days to 50% heading, spikelet or grain shape, pubescence, and overall phenotypic acceptability. DNA SNP genotyping was used to survey the EGMS lines for genes controlling the following traits: Wx (apparent amylose content), Alk (gel temperature), ACCase (Provisia herbicide tolerance), ALS (Clearfield herbicide tolerance), Pi-ta² (rice blast), GL (glabrous) and sd1 (plant height). An important consideration to advance or drop a line was ability of an EGMS line to produce high yielding hybrids in combination with male parents. These hybrids were evaluated in test cross nurseries and preliminary yield trials from 2017 to 2020. More details of this research are discussed below. The overall breeding pipeline for the selected EGMS lines is presented in Table 1.

TABLE 1
Breeding pipeline for EGMS lines from 2017 to 2020, H.
Rouse Caffey Rice Research Station, Crowley, Louisiana.

| Season | Planting | Selections |
|---|---|---|
| Summer 2017 | Single crosses: | |
| | 54 F_{4:5} sterile lines | 5 F_{5:6} sterile lines |
| | 43 F_{5:6} sterile lines | 7 F_{6:7} sterile lines |
| | 3 F_{6:7} sterile lines | |
| | Topcrosses: (Three-way) | |
| | 68 F_{3:4} sterile lines | 8 F_{4:5} sterile lines |
| | 49 F_{4:5} sterile lines | 8 F_{5:6} sterile lines |
| | 19 F_{5:6} sterile lines | |
| | 2 F_{6:7} sterile lines | |
| Summer 2018 | 59 Sterile lines | 24 Sterile lines |
| Summer 2019 | 18 Sterile lines | 12 Sterile lines |
| Summer 2020 | 8 Sterile lines | 5 Sterile lines |

### Environment-Sensitive Genic Male Sterility (EGMS)

During each of the 2017 to 2020 summer planting seasons, EGMS lines were planted in our Observation Nursery (ON) with a Hege90 planter in 2-meter rows, with a 0.025 m row width. The number of rows per line varied with seed availability. During the 2020 Summer planting season, approximately 3,420 EGMS lines that had been identified during previous years' development and evaluation were planted for testing and screening. These materials had been developed from two single crosses between 2009S sister lines and BASF's QPE-resistant O. indica lines: 13S-707-2/BASF 1-15, and 13S-615-2/BASF 1-14.

To test the pollen sterility of individual plants, unopened spikelets were sampled just before anthesis and evaluated under a microscope. At least six spikelets were collected from each plant, placed in a 96-well PCR microtiter plate with 150 μL of 1% iodine potassium iodide (IKI) solution prepared by dissolving 1 g of iodine and 2 g of potassium iodide in 100 mL of water. All anthers were removed using forceps, crushed on a slide by a bent needle, and observed under a microscope. The entire slide was scanned and pollen sterility count in three random fields was recorded. Unstained or irregularly shaped pollen grains were classified as sterile, while stained, round pollen grains were classified as fertile. Only those plants with 100% sterility in this assay were selected to advance in the breeding pipeline.

All selected male-sterile plants were crossed with pollinators to produce experimental hybrids; except for 2 to 4 plants that were cut back to the soil line and placed in pots in a greenhouse for ˜two weeks before transfer to a growth chamber with conditions conducive to reversion to fertility (22° C. night temperature, 28° C. day temperature, 10 hr. daylength) for 10 to 14 days. Plants were then transferred back to the greenhouse under summer temperature and daylength conditions to produce seed.

### Agronomic Characteristics and SNP Genotyping of the EGMS Lines

QPE-resistant EGMS lines in the 2020 EGMS ON were evaluated for emergence, days to 50% heading, spikelet or grain shape, and over-all phenotypic acceptability. Heading date of each EGMS line was observed from days of emergence to 50% heading. Leaf blade pubescence was determined based on visual inspection, and by rubbing fingers from the tip downward on the leaf surface. Grain was classified based on the length-to-width ratio of rough rice grains: long (above 3.4), medium (2.3-3.3), and short (below 2.2:1). The overall phenotypic acceptability (PAcp) of the rice plants was rated as 1 (excellent), 3 (good), 5 (fair), 7 (poor), or 9 (unacceptable) based on IRRI criteria. Materials were genotyped using Kompetitive allele specific PCR (KASP) for SNP trait markers.

### Results and Discussion

### Sterility Ratings of 2020 EGMS Observation Nursery (ON) Lines

The plants in the 2020 EGMS ON were sampled and observed under the microscope for pollen sterility evaluation before use as parents in test crosses. Only those plants initially assayed with completely sterile pollen grains (100% sterile) were selected to advance in the breeding pipeline. A number of partially fertile plants were nevertheless identified in the EGMS lines, suggesting some segregation for sterility was still ongoing. Some 2-4 plants with sterile pollen grains had been removed from the field and ratooned to produce purified EGMS seeds in the greenhouse under suitable conditions. At maturity EGMS plants with 30% or more seed set were selected as the source for the next generation of EGMS lines. All selected male sterile lines were used in test crosses to produce experimental hybrids for yield tests, described below.

### Agronomic Characterization of 2020 EGMS Observation Nursery (ON) Lines

As shown in Table 2, days from emergence to 50% heading (DTH) for the EGMS lines were 86 to 94 days in 2020 field plots, considered intermediate to late maturing. The lines had good to excellent phenotypic acceptability rating (PAcp). Sister lines 16HT 498-2-4 and 16HT 498-2-7 were classified as intermediate maturing lines with a mean of 86 DTH, average plant height of 64 cm, and PAcp rating of 1. EGMS Line 16HT 195-2 was identified as late maturing with a mean of 94 DTH, 78 cm plant height, and a phenotypic acceptability (PAcp) rating of 3. Three other sister lines designated as EGMS line 16HT695-1-3, 16HT695-1-6, and 16HT695-1-8 were classified as intermediate maturing lines with a mean of 89 DTH, average plant height of 81 cm, and PAcp rating of 3. The selected lines were short, with heights of 61 cm to 82 cm, which is desirable for a female parent for better pollination by a taller male parent. All EGMS lines were classified as pubescent (hairy) based on visual inspection. Two additional EGMS-lines, 16HT 397-1-6 and 16HT 489-1-6, were selected based on 2020 test cross performance of corresponding hybrids. 16HT 397-1-6 and 16HT 489-1-6 were both classified as intermediate maturing with a mean of 86 and 88 DTH, respectively, a plant height of 73 cm, and a PAcp rating of 3.

TABLE 2
Agronomic characteristics of eight QPE-resistant EGMS lines. Summer 2020,
H. Rouse Caffey Rice Research Station, Crowley, Louisiana.

| | | | | Days to | | | |
|---|---|---|---|---|---|---|---|
| | | | | 50% | Plant | | |
| Designation/ | | Seed_ | Seed | Heading | Height | | |
| Pedigree | Cross | I.D. | Source | (DTH)ª | (cm)^{b} | LBP^{c} | PAcp^{d} |
| 13TC 376-20-1-2 | 13S-707-2/ | 16HT | 17S1 | 94 | 78 | Pub | 3 |
| F7 | BASF 1-15 | 195-2 | 146-2 | | | | |
| 13TC 376-15-2- | 13S-707-2/ | 16HT | 18S2 53- | 86 | 66 | Pub | 1 |
| 4-F6 | BASF 1-15 | 498-2-4 | 3 | | | | |
| 13TC 376-15-2- | 13S-707-2/ | 16HT | 18S2 56- | 86 | 61 | Pub | 1 |
| 4-F6 | BASF 1-15 | 498-2-7 | 1 | | | | |
| 13TC 663-11-1- | 13S-615-2/ | 16HT | 18S1 109 | 89 | 82 | Pub | 3 |
| 4-F5 | BASF 1-14 | 695-1-3 | | | | | |
| 13TC 663-11-1- | 13S-615-2/ | 16HT | 18S2 93 | 89 | 79 | Pub | 3 |
| 4-F5 | BASF 1-14 | 695-1-6 | | | | | |
| 13TC 663-11-1- | 13S-615-2/ | 16HT | 18S1 140 | 89 | 82 | Pub | 3 |
| 4-F5 | BASF 1-14 | 695-1-8 | | | | | |
| 13TC 624-7-1- | 13S-210-2/ | 16HT | 19S2 140 | 86 | 73 | Pub | 3 |
| 1-F5⁺ | BASF 1-15 | 397-1-6 | | | | | |
| 13TC 376-15-1- | 13S-707-2/ | 16HT | 19S2 142 | 88 | 73 | Pub | 3 |
| 2-F5⁺ | BASF 1-15 | 489-1-6 | | | | | |

^{a}DTH = Average number of days from seedling emergence to 50% heading
^{b}Plant height = Height at maturity in centimeters from soil line to tip of longest panicle.
^{c}LBP = Leaf blade pubescence: Pub: Pubescent, Glab: Glabrous
^{d}PAcp = Phenotypic Acceptability: Excellent: 1, good: 3, fair: 5, poor: 7, unacceptable: 9 (International Rice Research Institute, 2013).
⁺Two additional EGMS-lines selected based on 2020 test cross (hybrids) performance, agronomic data from Summer 2019.

### DNA Marker Profiles of EGMS Observation Nursery (ON) Lines

As shown in Table 3, the EGMS lines generally were as homozygous for low-intermediate AAC (apparent amylose content). The single exception was 16HT 195-2, which was homozygous for the high AAC allele. Six lines were homozygous for the preferred intermediate gelatinization temperature (GT) (16HT 195-2, 16HT 498-2-4, 16HT 695-1-3, 16HT 695-1-6, 16HT 695-1-8 and 16HT397-1-6), while lines 16HT 498-2-7 and 16HT489-1-6 were homozygous for a low GT allele.

All lines were homozygous for the PV herbicide resistance genotype; except that 16HT 498-2-7 exhibited a homozygous conventional genotype (WT:WT). None of the EGMS lines carried the Pita-2 gene for blast resistance. The EGMS lines had a homozygous genotype for pubescence, which was confirmed by the presence of hair on the leaf surface. These observations had been expected, as none of the parents used in developing these EGMS lines carried the Pi-ta² resistant allele, nor the Glr1 glabrous allele. All selected EGMS lines contained the semidwarf sd1 allele for height, which was associated with a short plant height.

Three of the EGMS lines, 16HT 195-2, 16HT 498-2-4, and 16HT 695-1-3, were shown in 2019 Test cross plots described below to produce good hybrid combinations, comparable to commercial hybrids, with yields superior to inbred checks. EGMS lines 16HT 397-1-6 and 16HT 489-1-6 were selected based on performance of their hybrids in 2020 Test cross and Hybrid Preliminary Yield Trials. These five EGMS lines are currently planted for further purification and evaluation at the LSU AgCenter Plant Material Facility. Individual plant leaf samples were randomly collected for each line and submitted for SNP genotyping of trait and genome wide markers. Plants were further evaluated in 2021 summer field plots and in the Puerto Rico winter nursery.

These EGMS lines will be used in developing QPE-resistant two-line hybrids for the rice industry in Louisiana and elsewhere. The lines will be re-characterized each year to monitor and select their fertility alteration regimes, as these properties can drift over time if not affirmatively monitored and selected for.

TABLE 3
DNA marker profiles of QPE-resistant EGMS lines. Summer 2020, H. Rouse Caffey
Rice Research Station, Crowley, Louisiana.

| | | Seed | | Alk | Wx Ex10 | ALS | ACCase | Pita-2 | SD1 |
|---|---|---|---|---|---|---|---|---|---|
| Designation | Cross | I.D. | GLR1^{a} | (GT)^{b} | (AAC)^{c} | (CL)^{d} | (PV)^{e} | (blast)^{f} | (s.dwarf)^{g} |
| 13TC 376-20-1 | 13S-707- | 16HT | Pub:Pub | IGT:IGT | Hgh: Hgh | WT:WT | PV:PV | Sus:Sus | SH: SH |
| 2-F7 | 2/BASF 1-15 | 195-2 | | | | | | | |
| 13TC 376- | 13S-707-2/ | 16HT | Pub:Pub | IGT:IGT | Std:Std | WT:WT | PV:PV | Sus: Sus | SH: SH |
| 15-2-4-F6 | BASF 1-15 | 498-2 | | | | | | | |
| 13TC 376- | 13S-707-2/ | 16HT | Pub:Pub | LGT:LGT | Std:Std | WT:WT | WT:WT | Sus: Sus | SH: SH |
| 15-2-4-F6 | BASF 1-15 | 498-2-7 | | | | | | | |
| 13TC 663- | 13S-615- | 16HT | Pub:Pub | IGT:IGT | Std:Std | WT:WT | PV:PV | Sus: Sus | SH:SH |
| 11-1-4-F5 | 2/BASF 1-14 | 695-1-3 | | | | | | | |
| 13TC 663- | 13S-615-2/ | 16HT | Pub:Pub | IGT:IGT | Std:Std | WT:WT | PV:PV | Sus: Sus | SH: SH |
| 11-1-4-F5 | BASF 1-14 | 695-1-6 | | | | | | | |
| 13TC 663- | 13S-615-2/ | 16HT | Pub:Pub | IGT:IGT | Std:Std | WT:WT | PV:PV | Sus: Sus | SH:SH |
| 11-1-4-F5 | BASF 1-14 | 695-1-8 | | | | | | | |
| 13TC 624-7- | 13S-210-2/ | 16HT | Pub:Pub | IGT:IGT | Std:Std | WT:WT | PV:PV | Sus: Sus | SH:SH |
| 1-1-F5⁺ | BASF 1-15 | 397-1-6 | | | | | | | |
| 13TC 376- | 13S-707-2/ | 16HT | Pub:Pub | LGT:LGT | Std:Std | WT:WT | PV:PV | Sus: Sus | SH:SH |
| 15-1-2-F5⁺ | BASF 1-15 | 489-1-6 | | | | | | | |

^{a}Glabrousl gene: glabrous 1 SNP profile; Pub = pubescent, Glb = glabrous
^{b}Alkali gene: GT = gelatinization temperature; LGT = low gelatinization temperature, IGT = intermediate gelatinization temperature
^{c}Waxy gene: Exon10 SNP profile; AAC = apparent amylose content, Hgh = high AAC, Std = low to intermediate AAC
^{d}ALS = Acetolactate synthase gene: ALS-resistance; CL = Clearfield resistant, WT = wild type
^{e}ACCase = Acetyl coenzyme A carboxylase gene: QFE-resistance; PV = Provisia resistant, WT = wild type/PV susceptible
^{f}Pita-2 gene: Rice blast resistance; Sus = susceptible, Res = resistant
^{g}sd-1 gene: semidwarf SNP profile; SH = short, TL = tall
⁺Two additional EGMS-lines selected based on 2020 test cross (hybrids) evaluation

### Two-Line Rice Hybrids Resistant to the Herbicide Quizalofop-P-Ethyl

Commercial production of Clearfield® inbred varieties in Louisiana decreased from 45% in 2017 to 34% in 2019. This reduction was primarily due to the rise in imidazolinone-resistant weedy rice. Natural outcrossing has occurred between Clearfield® varieties and red rice, leading to the development of imidazolinone resistant weed in commercial fields. The development of Quizalofop-P-Ethyl (QPE) herbicide tolerant lines such as PVL01, PVL02, and PVL03 have helped efforts towards a long-term strategy for stewardship of the Clearfield® and technologies. The development of Quizalofop-P-Ethyl (QPE)-resistant hybrids with high grain yield, desirable agronomic traits, and excellent grain quality will further assist in these efforts.

### Materials and Methods

### Plant Materials Used in Producing QPE-Resistant Hybrids

The QPE-resistant, environmentally sensitive genetic male sterility system (EGMS) lines described above, along with selected male pollinator lines were used to produce a total of 550 distinct candidate hybrids that were evaluated in test cross nurseries and preliminary yield trials from 2018 to 2020. The pollinators comprised 50 QPE-resistant inbred lines from the LSU AgCenter as well as 46 conventional inbreds from southern U.S. rice breeding germplasms. Detailed listings of the pollinators, along with their agronomic traits and SNP marker profiles, can be found in the priority application, U.S. Ser. 63/178,591.

Test Cross Nursery Evaluation from 2018 to 2020

A total of 229 experimental hybrids originated from 2017 crosses were evaluated in the 2018 test cross field plots. Another 129 hybrids from 2018 crosses were evaluated in the 2019 Test cross nursery, and 192 hybrids from 2019 crosses were evaluated in the 2020 test crosses. Ten commercial hybrids and inbreds were used in the nurseries each year for comparison with the experimental hybrids. The number of rows per experimental hybrid varied with seed availability. Approximately 30 to 50 seeds of experimental hybrid per row were typically planted in the second to third week of March of 2018 and 2020 with a Hege 90 Magazine planter in 2-meter row length by 0.025-meter row width. Plot management followed standard practices for direct seeded rice in Southwest Louisiana.

In 2019, experimental hybrids grown in the greenhouse were transplanted to field plots 24 days after planting. Fifteen single seedlings per test hybrid were transplanted in a row with 20 cm spacing between seedlings. Data were recorded for number of days to emergence, days to 50% heading, plant height at maturity, lodging and grain type. Grain yield was determined after the whole row for each entry was harvested and grains were dried to 12% moisture content. Due to increased availability of candidate hybrid seed, the 2020TC test was laid out in a randomized complete block design (RCBD) with three replications (one row/rep/hybrid). Yield data were analyzed by Analysis of Variance and Fisher's Least Significant Difference at α=0.05. DNA SNP genotyping was completed through Kompetitive allele specific PCR (KASP) to survey the 2019TC and 2020TC hybrids for genes controlling the following traits: Wx (apparent amylose content), Alk (gel temperature), ACCase (Provisia), GL (glabrous) and sd1 (plant height).

### Hybrid Breeding Preliminary Yield Trial

A total of 37 QPE-resistant hybrids from crosses with 25 g of seeds were included in the 2020 Hybrid Breeding Preliminary Yield Trial (HYBPY) with plot size of 1.7 m×4.8 m (8.2 m²) per entry with one replication. The HYBPY comprised 136 experimental hybrids including conventional 3-line hybrids and 2-line hybrids with Clearfield and Provisia herbicide resistance. Thirteen hybrid and inbred varieties were used as checks for the experiment. The yield trial was planted on Apr. 4, 2020 at the LSU AgCenter South Farm, Crowley, Louisiana. Plot management followed standard practices for direct seeded rice in Southwest Louisiana. Data were recorded for number of days to emergence, days to 50% heading, grain type, plant height and lodging rate. Adjusted grain yield was computed using plot grain weight and moisture content data measured during harvesting using the Wintersteiger plot combine harvester.

### QPE Herbicide Treatment Experiment

To compare experimental hybrids containing one or two copies of the PV resistance gene, a greenhouse experiment was conducted in July 2020 with 13 experimental hybrids and four variety checks. Experimental hybrids were selected based on PV gene marker profiles of the corresponding parents. Three of the hybrids contained two copies from both parents; and six hybrids contained one copy from one parent. Four hybrids with no PV resistance gene were included in the test. Rice varieties PVL01 and PVL02 as resistant checks carried two copies of the resistance gene; whereas CL153 and Cheniere carried no PV resistance alleles. Seedlings grown in flats were treated at the 2-3 leaf stage with Provisia-BASF herbicide at a 1× rate equivalent to 120 g ai ha⁻¹ (Set I) and a 2× rate equivalent to 240 g ai ha⁻¹ (Set II). Set III contained check varieties PVL01, PVL02, CL153, and Cheniere, that served as controls without PV herbicide treatment. Responses of seedlings to herbicide applications were recorded 4 to 18 days after treatment on a scale where those that survived were rated resistant (R) while those that died were rated susceptible (S).

### Results and Discussion

### 2018 Test Cross Nursery Evaluation

The two highest yielding commercial hybrids in the 2018TC test were CLXL745 with 13026 kg/ha and XP760 with 12493 kg/ha (Table 4). Both hybrids produced long grains. The CLXL745 headed out earlier, at 79 days, and was more susceptible to lodging than was XP 760. Experimental hybrid 16HT195-2/14PVL038 produced comparable yields as the two commercial checks, with 13745 kg/ha. This hybrid produced long grains, was 91 days to 50% heading (intermediate to late maturity), and did not lodge with a height of 132 cm. Three more experimental hybrids produced more than 10 t/ha yield 16HT1642-1/17PV029 (10975 kg/ha), 16HT491-3/Diamond (10655 kg/ha), and 16HT1380-2/14PVL038-3 (10575 kg/ha). All were long grain type, intermediate maturity, comparable height to the hybrid variety checks and all were rated tolerant to lodging.

Four other experimental hybrids generated comparable yields relative to inbred and hybrid checks, ranging from 7392 kg/ha to 8604 kg/ha. The highest yielding commercially available inbred checks were IMI-herbicide resistant varieties CL 153 and CL 111, each with an identical yield of 7352 kg/ha. In summary, the best hybrid checks showed a mean yield of 10745 kg/ha and an 11% yield advantage over the experimental hybrids, which had a mean yield of 9680 kg/ha. On the other hand, the experimental hybrids had a 31% yield advantage over the best inbred checks.

TABLE 4
Grain yield, days to heading, plant height and lodging rate of
top experimental hybrids and checks from 18TC trial. Summer 2018,
H. Rouse Caffey Rice Research Station, Crowley, Louisiana.

| | Grain | | Days | Plant | |
|---|---|---|---|---|---|
| | yield | Grain | to 50% | Height | Lodging |
| Pedigree | (kg/ha)^{a} | Type^{b} | Heading^{c} | (cm)^{d} | Rate^{e} |
| 16HT195-2/14PVL038 | 13745 | L | 91 | 132 | 1.5 |
| CLXL 745 | 13026 | L | 79 | 114 | 3.5 |
| XP 760 | 12493 | L | 90 | 127 | 1.0 |
| 16HT1642-1/17PV029 | 10975 | L | 85 | 114 | 1.5 |
| 16HT491-3/Diamond | 10655 | L | 87 | 132 | 1.5 |
| 16HT1380-2/14PVL038-3 | 10575 | L | 87 | 120 | 1.5 |
| Gemini 214 CL | 9696 | L | 83 | 127 | 1.5 |
| 16HT1638-2/Diamond | 8604 | L | 88 | 116 | 1.5 |
| 16S 172-1/17RB481 | 7925 | L | 88 | 125 | 1.5 |
| XP753 | 7765 | L | 82 | 109 | 1.0 |
| 16HT202-4/17PV029 | 7565 | L | 79 | 133 | 3.0 |
| 16HT1642-1/RU1602140 | 7392 | L | 75 | 115 | 3.0 |
| CL153 | 7352 | L | 85 | 102 | 1.0 |
| CL111 | 7352 | L | 83 | 108 | 1.0 |

Mean Yield of Hybrid Checks: 10745 kg/ha
Mean Yield of Experimental Hybrids: 9680 kg/ha
Mean Yield of Inbred Checks: 7352 kg/ha
^{a}Adjusted grain yield from 1 row plot at 12% moisture content
^{b}Grain classifications by length to with ratio: L = long (3.4:1 and more), M = medium (2.3-3.3:1), and S = short (2.2:1 or less)
^{c}Average number of days from emergence to 50% heading
^{d}Plant height = distance in cm from soil to tip of longest panicle, taken at maturity.
^{e}Lodging rate: 1.0 = Strong, no bending; 3.0 = moderately strong, most plants lodging; 5.0 = Intermediate, most plants moderately bending); 7 = Weak, most plants nearly flat; 9 = very weak, all plants are flat

### 2019 Test Cross Nursery Evaluation

The best performing experimental hybrid was 16HT498-2-7/RU1702131, with 14054 kg/ha, long grains, 84 days to 50% heading (intermediate maturity), 106 cm in height and 1.5 lodging rate (Table 5). It was followed by 16HT695-1-8/17PV034 (13690 kg/ha), 16HT498-2-7/LAKAST (13421 kg/ha), and 16HT498-2-7/RU1601010 (13114 kg/ha). All were comparable to the highest yielding commercial hybrid check XP753, with 12403 kg/ha, long grains, 85 days to 50% heading (intermediate maturity), 107 cm in height and 1.0 lodging rate. Moreover, the experimental hybrids produced higher head rice yields at 60% vs. XP753 at 51%.

Six more experimental hybrids produced more than 10 t/ha yield: 16HT498-2-4/17PV001 (11597 kg/ha), 16HT1380-2/RU1702134 (10886 kg/ha), 16HT498-2-4/17PV013 (10752 kg/ha), 16HT1380-2/17PV025 (10637 kg/ha), 16HT 195-2/PV038-2 (10368 kg/ha), and 16HT695-1-6/ru1703147 (10253 kg/ha). These hybrids headed at 77 to 84 days (early to intermediate maturity), had long grain type, short to moderate height of 96 cm to 104 cm (except for 16HT 195-2/PV038-2 at 115 cm), and 1.0 to 1.5 lodging rate. They also showed good head rice yields of 56 to 6300. All experimental hybrids were comparable to the second highest-yielding hybrid P 760, which produced 10598 kg/ha, had long grains, 111 cm height, 1.5 lodging rate, and 600 head rice yield.

TABLE 5
Grain yield, days to heading, plant height, lodging rate and milling quality of top experimental
hybrids and checks from 19TC trial. Summer 2019, H.
Rouse Caffey Rice Research Station, Crowley, Louisiana.

| | Grain | | Days | Plant | | Milling % |
|---|---|---|---|---|---|---|
| | yield | Grain | to 50% | Height | Lodging | (whole/ |
| Pedigree | (kg/ha)^{a} | Type^{b} | Heading^{c} | (cm)^{d} | Rate^{e} | total)^{f} |
| 16HT498-2- | 14054 | L | 84 | 106 | 1.5 | 61/70 |
| 16HT695-1- | 13690 | L | 83 | 102 | 1.5 | 61/69 |
| 16HT498-2- | 13421 | L | 85 | 123 | 3.5 | 60/69 |
| 16HT498-2- | 13114 | L | 82 | 113 | 1.5 | 60/71 |
| XP753 | 12403 | L | 85 | 107 | 1.0 | 51/69 |
| 16HT498-2- | 11597 | L | 79 | 104 | 1.5 | 60/69 |
| 16HT1380- | 10886 | L | 77 | 99 | 1.5 | 59/69 |
| 16HT498-2- | 10752 | L | 78 | 100 | 1.5 | 59/68 |
| 16HT1380-2/17PV025 | 10637 | L | 77 | 98 | 1.0 | 56/70 |
| XP760 | 10598 | L | 87 | 111 | 1.5 | 60/69 |
| 16HT 195-2/PV038-2 | 10368 | L | 83 | 96 | 1.0 | 63/70 |
| 16HT695-1- | 10253 | L | 84 | 115 | 1.5 | 60/69 |
| CLXL745 | 10099 | L | 78 | 96 | 3.5 | 64/71 |
| 16HT1499-1/PV021 | 9638 | L | 74 | 102 | 3.0 | 60/69 |
| 16HT498-2-7/PV021 | 9466 | L | 74 | 100 | 1.5 | 61/67 |
| (Short Selection) | | | | | | |
| 16HT727-2- | 9446 | L | 77 | 98 | 1.5 | 58/69 |
| 16HT1564-1- | 9254 | L | 76 | 88 | 1.0 | 60/71 |
| 16HT1499-1/17PV013 | 9216 | L | 77 | 87 | 1.0 | 49/69 |
| 16HT1499-1/17PV063 | 9101 | L | 85 | 102 | 1.5 | 61/70 |
| 16HT498-2- | 8986 | L | 77 | 103 | 1.5 | 63/69 |
| RT7311CL | 8794 | L | 84 | 95 | 3.0 | 54/71 |
| 16HT1499-1/17PV033 | 8755 | L | 82 | 94 | 1.5 | 59/70 |
| 16HT695-1- | 8717 | L | 77 | 94 | 1.5 | 63/69 |
| 16HT489-1- | 8602 | L | 78 | 101 | 1.5 | 63/69 |
| 16HT1524-1/17PV012 | 8582 | L | 78 | 92 | 3.5 | 58/70 |
| 16HT1642-1/17PV027 | 8506 | L | 75 | 94 | 3.5 | 59/66 |
| CL153 | 8429 | L | 82 | 93 | 1.0 | 58/70 |
| 16HT695-1- | 8410 | L | 78 | 93 | 3.0 | 59/69 |
| 16HT695-1- | 8410 | L | 76 | 86 | 3.0 | 55/63 |
| 16HT1380-2- | 8410 | L | 79 | 101 | 3.0 | 53/69 |
| 16HT1380-2- | 8410 | L | 81 | 95 | 1.5 | 56/69 |
| 4/RU1601010 | | | | | | |

Mean Yield of Hybrid Checks: 10474 kg/ha
Mean Yield of Experimental Hybrids: 10026 kg/ha
Mean Yield of Inbred Check: 8429 kg/ha
^{a}Adjusted grain yield from 1 row plot at 12% moisture content
^{b}Grain type classification by length to with ratio: L = long (3.4:1 and more), M = medium (2.3-3.3:1), and S = short (2.2:1 or less)
^{c}Average number of days from emergence to 50% heading.
^{d}Plant height = distance in cm from soil to tip of longest panicle, taken at maturity.
^{e}Lodging rate: 1.0 = Strong, no bending; 3.0 = moderately strong, most plants lodging; 5.0 = Intermediate, most plants moderately bending); 7 = Weak, most plants nearly flat; 9 = very weak, all plants are flat.
^{f}Milling quality: Ratio of % head (whole) rice yield to % milling (total) rice yield.

An additional 16 hybrids showed grain yields from 8410 kg/ha to 9638 kg/ha, comparable to hybrid and inbred checks. The highest yielding inbred check was CL153, with 8429 kg/ha. The three top commercial hybrids in this test had a mean yield of 10474 kg/ha, with a 5% yield advantage over the best experimental hybrids (10026 kg/ha). On the other hand, the best experimental hybrids had a 19% yield advantage over the best inbred check.

Details of the DNA marker profiles of the top yielding experimental hybrids with comparable yields to check varieties can be found in priority application U.S. Ser. 63/178,591. Two of the top 10 experimental hybrids (16HT1380-2/RU1702134; 16HT1380-2/17PV025) were observed in the field as glabrous, confirmed by their homozygous Glb:Glb genotype. The pollinators used were both glabrous, as was the EGMS line 16HT1380-2 which was one of the few glabrous QPE-resistant EGMS parents in this material. Three other EGMS lines were identified as glabrous based on marker genotypes of the hybrids, although they were expected to be pubescent. These results may be due to segregation or mixtures in the material. All other experimental hybrids were pubescent.

Nine of the top 10 experimental hybrids showed intermediate GT, with a homozygous Int:Int genotype; while the remaining hybrid was heterozygous for low-intermediate GT. Two of the top 10 hybrids showed intermediate AAC (16HT1380-2/RU1702134 and 16HT1380-2/17PV025); one hybrid (16HT 195-2/PV038-2) exhibited high AAC; and another (16HT695-1-8/17PV034) had low AAC. The remaining hybrids were heterozygous for the low-intermediate AAC marker. All hybrids contained at least one copy of the ACCase (PV) gene, except for two hybrids (16HT695-1-8/17PV034 and 16HT727-2-4/RU1702134) that carried no PV resistance alleles.

A newly designed sd-1 SNP marker for KASP genotyping was used to detect the sd-1 semidwarf allele in the 2019TC hybrids. Based on the genotype profiles, all experimental hybrids, except for two, were homozygous (SH:SH) for the short sd-1 allele. The actual plant height of the hybrids and the genotype profiles for sd-1 generally corresponded to the observed heights of 86 cm to 106 cm, with a mean height of 97 cm. However, the association was poor for two hybrids 16HT498-2-7/LAKAST (123 cm) and 16HT498-2-7/RU1601010 (113 cm) having the same female parent. Only two hybrids heterozygous for these markers (SH:TL) were scored as tall (16HT498-2-7/RU1702131 (106 cm) and 16HT695-1-6/RU1703147 (115 cm).

### 2020 Test Cross Nursery Evaluation

There were significant differences among mean yields of the 200 hybrids and among blocks, which was not unexpected due to small plot size inherent in test cross nurseries. The coefficient of variation (CV) in the experiment was also relatively high at 16%. The top yielding hybrid for the 2020 Test cross Nursery was RT7321, a RiceTec FullPage™ IMI-tolerant hybrid variety with 12013 kg/ha. It was followed by four QPE-resistant experimental hybrids: 16HT 489-1-6/RU1601010 with 10816 kg/ha, 16HT 489-1-6/Diamond with 10650 kg/ha, 16HT 397-1-6/Maybelle with 10509 kg/ha, and 16HT 397-1-6/RU1702131 with 10202 kg/ha. Based on the LSD-test, the yields of these top five hybrids were not significantly different from one another. However, RT7321 was significantly different from the remaining hybrids in this trial. The yield of the top experimental hybrid, 16HT 489-1-6/RU1601010 (10816 kg/ha), was not significantly different from the next 11 high-yielding experimental hybrids, with yields from 8934 kg/ha to 10650 kg/ha. Yields of the 14 remaining hybrids were 8275 kg/ha to 8902 kg/ha, including CLXL 745 (8493 kg/ha) and Gemini 214 CL (8275 kg/ha). Experimental hybrid yields from 8512 kg/ha to 10202 kg/ha were not significantly different from the hybrid check CLX745.

TABLE 6
Analysis of variance of grain yield data from 2020
Test cross Nursery. Summer 2020, H. Rouse Caffey
Rice Research Station, Crowley, Louisiana.

| Source of | Degrees of | Sum of | Mean | F | |
|---|---|---|---|---|---|
| Variation | Freedom | Squares | Square | value | Pr(>F) |
| Treatment | 199 | 1.107e+09 | 5563727 | 3.979 | <2e−16 *** |
| (Hybrids) | | | | | |
| Replication | 2 | 2.145e+07 | 10724875 | 7.671 | 0.000539 *** |
| (Blocks) | | | | | |
| Error | 398 | 5.565e+08 | 1398166 | | |

Treatment Means: Rep1 = 7528; Rep 2 = 7120; Rep 3 = 7135
Grand Mean: 7260.96
Signif. codes:
*** <0.001
C.V.: 16.2849

There were differences for mean days to 50% heading (DTH) among the 200 hybrids and among blocks. The coefficient of variation (CV) was relatively low at 2%. Based on the LSD-test, the DTH of the four highest-yielding experimental hybrids did not differ from that of RT7321, except for hybrid 16HT 489-1-6/Diamond.

TABLE 7
Grain yield, days to 50% heading, and plant height of top hybrids from 2020 Test cross
Nursery. Summer 2020, H. Rouse Caffey Rice Research Station, Crowley, Louisiana.

| | Grain Yield | Days to 50% Heading | Plant Height |
|---|---|---|---|
| Pedigree | (t/ha)^{a} | (DTH)^{b} | (cm)^{c} |
| RT 7321FP | 12013⁺ a | 86.7⁺ lmnopqrst | 116.7⁺ hijklmn |
| 16HT 489-1-6/RU1601010 | 10816 ab | 88.7 fghijklmn | 116.7 hijklmn |
| 16HT 489-1-6/Diamond | 10650 abc | 89.7 cdefghijk | 119.3 efghi |
| 16HT 397-1-6/Maybelle | 10509 abcd | 88.0 hijklmnop | 124.0 cd |
| 16HT 397-1-6/RU1702131 | 10202 abcde | 87.7 ijklmnopq | 115.0 jklmnopqrs |
| 16HT 1651-1/RU1702131 | 9882 bcdef | 88.3 ghijklmno | 120.3 defgh |
| 16HT 397-1-6/RU1804175 | 9600 bcdefg | 90.0 cdefghij | 110.3 vwxyzABCDEF |
| 16HT 1651-1/14PVL038-1 | 9402 bcdefgh | 83.3 vwxyzABCD | 117.3 ghijklm |
| 16HT 397-1-6/PSDO | 9376 bcdefghi | 89.3 defghijkl | 114.3 lmnopqrstu |
| 16HT 498-2-4/RU1805198 | 9082 bcdefghij | 85.0 qrstuvwxy | 120.3 defgh |
| 16HT 498-2-4/17PV 034 | 9030 bcdefghijk | 88.7 fghijklmn | 106.3 GHIJKLMNOPQ |
| 16HT 498-2-4/RU1703147 | 8992 bcdefghijkl | 87.0 klmnopqrs | 115.3 jklmnopqr |
| 16HT 489-1-6/RU1802110 | 8934 bcdefghijklm | 87.7 ijklmnopq | 112.3 pqrstuvwxyzA |
| 16HT 498-2-4/RU1902222 | 8902 cdefghijklmn | 89.7 cdefghijk | 115.3 jklmnopqr |
| 16HT695-1-3/PSDO | 8896 cdefghijklmno | 88.3 ghijklmno | 117.7 ghijkl |
| 16HT 397-1-6/RU1801173 | 8813 cdefghijklmnop | 90.7 cdefgh | 114.0 lmnopqrstuv |
| 16HT 1380-2/RU1902026 | 8698 defghijklmnopq | 85.3 pqrstuvwx | 110.0 wxyzABCDEFG |
| 16HT 727-2-1/RU1902086 | 8691 defghijklmnopq | 90.3 cdefghi | 116.3 ijklmno |
| 16HT 731-1/RU1801173 | 8685 defghijklmnopq | 87.3 jklmnopqr | 109.0 zABCDEFGHIJ |
| 16HT 1630-1/RU1601010 | 8659 defghijklmnopqr | 85.7 opqrstuvw | 115.3 jklmnopqr |
| 16HT 695-1-3/PVL038 | 8608 efghijklmnopqrs | 83.3 vwxyzABCD | 104.0 NOPQRSTUVWX |
| 16HT 489-1-6/RU1902026 | 8582 efghijklmnopqrst | 84.3 stuvwxyzA | 116.0 ijklmnop |
| 16HT 733-1/ RU1601010 | 8570 efghijklmnopqrst | 86.3 mnopqrstu | 116.3 ijklmno |
| 16HT 498-2-4/PSDO | 8531 efghijklmnopqrstu | 89.0 efghijklm | 115.3 jklmnopqr |
| 16HT 397-1-6/RU1805198 | 8525 efghijklmnopqrstuv | 88.3 ghijklmno | 110.0 wxyzABCDEFG |
| 16HT195-2/RU1902222 | 8525 efghijklmnopqrstuvw | 89.7 cdefghijk | 123.3 cd |
| 16HT 489-1-6/17PV 034 | 8512 efghijklmnopqrstuvwx | 88.7 fghijklmn | 113.0 nopqrstuvwxy |
| CLXL 745(HybridCH_CL) | 8493 efghijklmnopqrstuvwxy | 84.7 rstuvwxyz | 109.3 yzABCDEFGHI |
| Gemini 214 (HybridCH_CL) | 8275 fghijklmnopqrstuvwxyz | 90.7 cdefgh | 122.7 cde |

Mean Yield LSD_{0.05} = 1898.036
Mean DTH LSD_{0.05} = 2.794129
Mean Plant height LSD_{0.05} = 3.905584
⁺Means followed by a common letter in the column are not significantly different by the LSD-test at the 5% level of significance.
LSD = Least significant difference at 5% level of significance.
^{a}Adjusted grain yield from 1 row plot at 12% moisture content.
^{b}Average number of days from emergence to 50% heading.
^{c}Plant height = distance in cm from soil to tip of longest panicle, taken at maturity.

TABLE 8
Analysis of variance for days to 50% heading from
2020 Test cross Nursery. Summer 2020, H. Rouse Caffey
Rice Research Station, Crowley, Louisiana.

| Source of | Degrees of | Sum of | Mean | | |
|---|---|---|---|---|---|
| Variation | Freedom | Squares | Square | F value | Pr(>F) |
| Treatment | 199 | 9484.5 | 47.66 | 15.731 | <2.2e−16 *** |
| (Hybrids) | | | | | |
| Replication | 2 | 1004.8 | 502.41 | 165.86 | <2.2e−16 *** |
| (Blocks) | | | | | |
| Error | 398 | 1205.8 | 3.03 | | |

Treatment Means: Rep1 = 86.740; Rep 2 = 85.005; Rep 3 = 83.575
Grand Mean: 85.10667
Signif. codes:
*** <0.001
C.V.: 2.045303

There were differences in plant height among the 200 hybrids, while there were no differences detected among blocks. The coefficient of variation (CV) was relatively low at 2%. Based on the LSD-test, the plant height of the four highest yielding experimental hybrids did not differ significantly from RT7321, except for hybrid 16HT 397-1-6/Maybelle.

TABLE 9
Analysis of variance of plant height data from 2020
Test cross Nursery. Summer 2020, H. Rouse Caffey
Rice Research Station, Crowley, Louisiana.

| Source of | Degrees of | Sum of | Mean | | |
|---|---|---|---|---|---|
| Variation | Freedom | Squares | Square | F value | Pr(>F) |
| Treatment | 199 | 32209 | 161.86 | 27.353 | <2e−16 *** |
| (Hybrids) | | | | | |
| Replication | 2 | 12 | 6.10 | 1.031 | 0.358 |
| (Blocks) | | | | | |
| Error | 398 | 2355 | 5.92 | | |

Treatment Means: Rep1 = 109/75; Rep 2 = 109.53; Rep 3 = 109.87
Grand Mean: 109.7133
Signif. codes:
*** <0.001
C.V.: 2.217693
LSD_{0.05} = 3.905584

Genotyping of high-yielding hybrids revealed SNPs at Exon 10 for high AAC for the rice waxy (Wx) gene. Two of the top yielding experimental hybrids; 16HT 397-1-6/RU1702131 and 16HT 397-1-6/PSDO, were both heterozygous for high AAC, while the rest were found to be homozygous for low-intermediate AAC.

Four hybrids had a desirable gelatinization temperature (GT): 16HT 489-1-6/RU1601010, 16HT 498-2-4/17PV 034, 16HT 489-1-6/RU1802110, and 16HT 498-2-4/RU1902222 and were heterozygous for low-intermediate GT, while the others had homozygous genotypes for intermediate GT.

All experimental hybrids were genotyped as pubescent, confirmed by the presence of trichomes on the leaf blade surface.

The sd1 SNP marker was not associated with the semi-dwarfing trait for some hybrids. For example, hybrid 16HT 397-1-6/Maybelle (20TC1-0191) was homozygous for the sd1 marker genotype, but the actual plant height was 124 cm, which is considered tall even for a hybrid. Similar observations were made for 16HT 489-1-6/RU1601010 (20TC1-0150) (117 cm), 16HT 1651-1/14PVL038-1 (20TC1-0048) (117 cm), 16HT695-1-3/PSDO (20TC1-0028) (118 cm), 16HT 489-1-6/Diamond (20TC1-0077) (119 cm), 16HT 1651-1/RU1702131(20TC1-0136) (119 cm), and 16HT 498-2-4/RU1805198 (20TC1-0045) (120 cm). Additional work is needed concerning the sd1 marker and plant height in rice hybrids.

### QPE Resistance of Experimental Hybrids

The high-yielding experimental hybrids were all classified as QPE herbicide resistant, due to presence of at least one copy of the dominant ACCase (PV) mutant gene, contributed by the QPE EGMS female parents. The two Provisia inbred varieties PVL01 and PVL02 contained two copies of the gene. However, field treatment with the PV herbicide to confirm resistance was not conducted initially, due to the limited number of seed available and potential problems with herbicide drift onto adjacent breeding materials. Instead, herbicide resistance was tested in the greenhouse.

Provisia inbred varieties PVL01 and PVL02 both survived the 1× rate and 2× rates of QPE treatment, as expected. Three experimental hybrids: 19S1 158-9/19XB1-87, 1952 164-17/19XB2-204, and 1952 140-7/19XB2-209 with two copies of the PV gene each, from both parents, also survived both rates of herbicide. Six hybrids derived from one resistant and one susceptible parent with a heterozygous PV:WT genotype also showed a resistant reaction to herbicide treatment. It has been previously reported that QPE-resistance is controlled by a dominant gene, with either one or two copies of the resistance allele giving a resistant phenotype.

### 2020 Hybrid Preliminary Yield Trial

Grain yield, grain type, days to 50% heading, plant height, and lodging rate of the top 35 hybrids in the 2020 Hybrid Breeding Preliminary Yield are shown in Table 10. Grain yields for the 15 QPE-resistant hybrids were 9224 to 11019 kg/ha, with a mean of 9703 kg/ha. QPE hybrid 16HT397-1-6/RU1601010 produced the highest yield at 11019 kg/ha, similar to yields for RT7521 FP and Gemini 214 CL. The three highest yielding QPE-resistant experimental hybrids all had 16HT397-1-6 as the EGMS female parent, indicating that this line has good combining ability. The six RiceTec hybrids in this test, with a mean yield of 9991 kg/ha, had a 3% yield advantage over the 15 QPE candidate hybrids.

Hybrids 16HT397-1-6/RU1601010 and 16HT397-1-6/RU1701105 had mean plant heights of 112 cm to 113 cm, and were rated as moderately resistant to lodging, similar to the RiceTec hybrids evaluated in this trial. Hybrid 16HT 397-1-6/Maybelle was taller, with mean plant height of 120 cm, and was rated as moderately susceptible to lodging. Days to 50% heading for the top QPE-resistant experimental hybrid 16HT397-1-6/RU1601010 were 81 days, while the two hybrids 16HT397-1-6/Maybelle and 16HT397-1-6/RU1701105 headed at 85 and 92 days, respectively, comparable to the RiceTec hybrid checks.

TABLE 10
Grain yield, grain type, days to 50% heading, plant height, and lodging rate of top
hybrids in the 2020 Hybrid Breeding Preliminary Yield (HYBPY) trial. Summer 2020, LSU
AgCenter South Farm, Crowley, Louisiana.

| | | | Grain | | Days | Plant | |
|---|---|---|---|---|---|---|---|
| | | | Yield | Grain | to 50% | Height | Lodging |
| Plot ID | Pop ID | Pedigree | (kg/ha)^{a} | Type^{b} | Heading^{c} | (cm)^{d} | Rate^{e} |
| 20HYBPY105 | 19DX0043 | 16HT 397-1-6/RU1601010 | 11019 | L | 81 | 113 | MR |
| 20HYBPY126 | Check | RT7521 FP | 10701 | L | 88 | 110 | MR |
| 20HYBPY125 | Check | GEMINI 214 CL | 10676 | L | 88 | 108 | MR |
| 20HYBPY073 | CLXL745 | CLXL745 | 10503 | L | 84 | 113 | MR |
| 20HYBPY072 | XP753 | XP753 | 10492 | L | 85 | 111 | MR |
| 20HYBPY104 | 19DX0166 | 16HT 397-1-6/RU1701105 | 10464 | L | 92 | 112 | MR |
| 20HYBPY087 | 19DX0038 | 16HT 397-1-6/Maybelle | 10048 | L | 85 | 120 | MS |
| 20HYBPY025 | 19LX0576 | 69S/1703190 | 10048 | L | ND | 120 | ND |
| 20HYBPY123 | Check | RT7321 | 10034 | L | ND | 112 | ND |
| 20HYBPY027 | 19LX0568 | 69S/1704083 | 10008 | L | ND | 119 | ND |
| 20HYBPY029 | 19LX0559 | 69S/CL151 | 9996 | L | ND | 129 | ND |
| 20HYBPY026 | 19LX0732 | 69S/1704077 | 9945 | L | ND | 122 | ND |
| 20HYBPY093 | 19 DX0040 | 16HT 397-1-6/RU1805198 | 9791 | L | 91 | 116 | MR |
| 20HYBPY101 | 19DX0141 | 16HT 733-1/17PV 034 | 9774 | L | 92 | 108 | MR |
| 20HYBPY116 | 19250F1-19_36 | 16SF1034-2/RU1702168 | 9772 | L | ND | 100 | ND |
| 20HYBPY107 | 19DX0142 | 16HT 733-1/17PV 066 | 9706 | L | 85 | 111 | MS |
| 20HYBPY089 | 19DX0047 | 16HT731-1/RU1805198 | 9618 | L | 92 | 111 | MS |
| 20HYBPY020 | 19LX0574 | 69S/1303184 | 9501 | L | ND | 108 | ND |
| 20HYBPY103 | 19DX0177 | 16HT 733-1/RU1701105 | 9495 | L | 88 | 122 | MR |
| 20HYBPY097 | 19DX0099 | 16HT 733-1/Diamond | 9425 | L | 91 | 123 | MR |
| 20HYBPY023 | 19LX0582 | 69S/1601067 | 9392 | L | ND | 131 | ND |
| 20HYBPY109 | 19DX0072 | 16HT 727-2-1/RU1805198 | 9379 | L | 87 | 108 | MR |
| 20HYBPY094 | 19DX0136 | 16HT 733-1/10HHB020 | 9355 | L | 88 | 102 | MR |
| 20HYBPY102 | 19DX0095 | 16HT 733-1/RU1805198 | 9316 | L | 88 | 105 | MS |
| 20HYBPY071 | CLXL745 | CLXL745 | 9297 | L | 84 | 117 | MR |
| 20HYBPY110 | 19DX0101 | 16HT 733-1/17PV013 | 9237 | L | 83 | 106 | MR |
| 20HYBPY086 | 19DX0132 | 16HT 733-1/PSDO | 9224 | L | 95 | 120 | MR |
| 20HYBPY117 | 19250F1-37_54 | 16SF1035-1/ | 9221 | L | ND | 120 | ND |
| | | (FRANCIS/5/LGRU/CLR | | | | | |
| | | 22/4/96020653CFX-29/AR | | | | | |
| | | 1142/LA 2031) | | | | | |
| 20HYBPY028 | 19LX0587 | 69S/Cheniere | 9185 | L | ND | 117 | ND |
| 20HYBPY030 | 19LX0561 | 69S/Cypress | 9132 | L | ND | 120 | ND |
| 20HYBPY127 | Check | RT7301 | 9125 | L | ND | 105 | ND |
| 20HYBPY022 | 19LX0566 | 69S/1501108 | 9111 | L | ND | 144 | ND |
| 20HYBPY124 | Check | CLXL745 | 9103 | L | 84* | 107 | MR |
| 20HYBPY067 | 19250F1-27_36 | 16SF1034-2/RU1702168 | 9103 | L | ND | 109 | ND |
| 20HYBPY114 | 19DX0033 | 16HT 695-1-3/RU1703147 | 9100 | L | 86 | 105 | MR |

^{a}Adjusted grain yield from 1 row plot at 12% moisture content
^{b}Grain type classification by length to with ratio: L = long (3.4:1 and more), M = medium (2.3-3.3:1), and S = short (2.2:1 or less)
^{c}Average number of days from emergence to 50% heading.
^{d}Plant height = Height at maturity in centimeters from soil line to tip of longest panicle.
^{e}Lodging Rate: Comparative estimate of resistance to lodging. Varieties rated as resistant can still lodge, especially under excessive levels of nitrogen.
Abbreviations:
ND = no data,
R = resistant,
MR = moderately resistant,
MS = moderately susceptible.

Susceptible checks Cheniere and CL153 both died after the QPE herbicide treatment. Three of the four hybrids that did not carry the PV gene, based on the SNP profile of both their parents (19S2 144-11/19XB2-30, 19S2 144-13/19XB2-34, and 19S2 149-1/19XB2-26), showed a heterozygous PV:WT genotype after leaf samples of the hybrids were analyzed. Thus, there was a mismatch of SNP profile analysis between the parents and the hybrids. However, they all died after herbicide treatment, the same reaction expected of those who did not carry the PV gene. This mismatch of marker analysis might have resulted from SNP genotyping errors or seed mixtures. To confirm the results, material from this experiment was treated again with PV herbicide on Feb. 10, 2021 at the same rates in a duplicate experiment. Future genotyping of key breeding material should include a minimum of three genotyping replications for each SNP to ensure consistent results.

Overall evaluation of the experimental hybrids from 2018 to 2020 demonstrated the ability of the QPE-resistant EGMS lines to produce high yielding, two-line hybrids with desired grain quality traits and good adaptation to local conditions. Selection of breeding material over four years resulted in the identification of the following five elite EGMS lines: 16HT489-1-6, 16HT397-1-6, 16HT498-2-4, 16HT695-1-3, and 16HT 195-2, showing good agronomic traits and desirable seed dimensions. Based on the performance of their hybrids in 2020 Hybrid Preliminary Yield Trial and Test cross Nurseries from 2018-2020, as well as their agronomic and DNA marker profile for target traits, the selected EGMS lines are grown in the greenhouse for additional purification and seed increase.

In addition to high yield, several QPE-resistant experimental hybrids in both the TC and HYBPY trials exhibited long grains, early to intermediate maturity, comparable height with hybrid checks, and moderate tolerance to lodging. In general, these experimental hybrids also exhibited better milling quality than the commercially available hybrids. The top overall hybrids (16HT 397-1-6/RU1601010, 16HT 397-1-6/RU1701105, and 16HT 397-1-6/Maybelle) from these trials will be reproduced and will be further evaluated in preliminary and multi-location yield trials.

Results of the herbicide resistance greenhouse test showed expected results, with the Provisia varieties (PVL01 and PVL02), each carrying two copies of the PV resistance gene, surviving both herbicide rates; while varieties without the resistance gene (Cheniere and CL153) were dead 18 days after herbicide treatment. The three experimental hybrids with two copies of the PV resistance gene also survived both rates of the Provisia herbicide treatment, as did the six hybrids with one copy of the gene. The four experimental hybrids lacking the ACCase (PV) resistance gene all died 18 days after treatment. However, three of these susceptible hybrids showed a heterozygous profile (sampled from hybrids planted in 2020 Test cross nursery) for the gene, suggesting they contained a single copy of the PV gene. This mismatch of the marker analysis between the parents and the hybrids may be due to SNP genotyping errors. Another possible cause is that the hybrid plants sampled for genotyping could be a seed mixture or seed from another hybrid. Duplicate greenhouse experiments will resolve these discrepancies. Additional tests of experimental hybrids will characterize herbicide response under field conditions.

The new QPE herbicide-resistant hybrids are competitive with the leading Provisia® commercial hybrids because of their high yield, QPE herbicide resistance and better milling quality. Seeds from the two-line hybrids can be produced at a lower price than for three-line hybrids. The selected EGMS lines, which are all classified as subspecies indica, can also be crossed with male pollinators classified as tropical japonica, to produce intersubspecific hybrids with high yields.

Seed Production Methods for Parental Lines and Hybrids, under Greenhouse and Field Conditions

Effective and cost-effective methods for production of parental lines and hybrid seed are important components for two-line hybrid breeding programs. Typically, pollinator lines are produced in the field and greenhouse just as for inbred varieties; while environmentally sensitive genetic male sterility system (EGMS) lines are maintained by continuous selection for critical temperature or photoperiod, and are regularly tested for pollen sterility and seed fertility. Purity of both EGMS and pollinator lines is important because they are used in producing candidate hybrids that are selected or dropped during the breeding pipeline. Factors that affect hybrid seed production include plant height, synchronization of flowering, exertion of panicles, isolation method, row ratio, rouging, and supplementary pollination. Spraying of gibberellic acid (GA₃) at an appropriate time can enhance seed set by inducing panicle and stigma exertion, plant height, and duration of floret opening. Supplementary pollination, for example shaking pollen parents with a bamboo stick, may be conducted at the peak period of anthesis to enhance seed set.

One method used for seed production is the “chimney” method: The male and female plants are both transferred to a 1 m² area just before flowering. The plants are then covered by a 1 m×1 m×1 m metal frame cubicle covered in muslin cloth to inhibit cross pollination. During the peak of anthesis (flowering), the pollinator (male) parents are shaken through an opening on top of the cubicle to induce pollination and increase seed setting. In a modification of the chimney method, 2 m high barriers cover three sides of a seed production plot, with a gap of 20 cm from the ground. The fourth, open side is covered by the barrier of a neighboring plot, leaving just enough room for use in cultural operations including supplementary pollination. An isolation-free method of seed production is easier to implement and may be better accepted in tropical countries: A plot of six rows of female parents is planted alternately with single rows of the male parents. Then the entire plot is surrounded by four rows of the same male parent, to serve as a natural barrier to pollen from adjoining plots.

### Materials and Methods

### Seed Increase of EGMS Lines by the Ratoon Method

A total of 128 pollen sterile plants from 14 EGMS lines were used in crosses with pollinator lines at the H. Rouse Caffey Rice Research Station in 2018. In 2019, 95 pollen-sterile plants from 11 EGMS lines were used in crosses. After harvesting hybrid seeds from individual EGMS plants, the plants were cut back near the base, and then transferred to and grown in the greenhouse for ˜15 days. During this time the ratooned plants were treated every seven days with Quadris fungicide at 6 mL/L to encourage rapid growth of new healthy tissue. Plants were then transferred to the HRCRRS growth chamber once the flag leaf of the primary tiller had completely emerged from the leaf sheath, exposing about one to two cm of the internode from the flag leaf collar to the second leaf node. The growth chamber was set for a 10-hour day, 25° C. during the day and 22° C. during the night for 10 to 14 days. Plants were then transferred to regular greenhouse conditions for a seed production target of 1 g per plant, equivalent to ˜50 seeds.

Seed Increase and Purification of EGMS lines in the Greenhouse Under Controlled Conditions.

With the availability in 2019 of the Plant Material Facility at the LSU Ben Hur Farm in Baton Rouge, Louisiana, seed increase and purification of EGMS lines was carried out for six promising EGMS lines in the Fall to Winter Season (October 2019-February 2020). These promising EGMS lines were selected based on performance of their hybrids in the 2019 Test cross Nursery (TN). The selected lines were designated 16HT695-1-6, 16HT695-1-8, 16HT695-1-3, 16HT195-2, 16HT498-2-4, and 16HT498-2-7.

Wooden benches 2.4 m long, 1.2 m wide, and 0.25 m deep were covered with plastic and filled with greenhouse-sterilized soil mixed with sphagnum organic peat moss (Majestic Earth) in a ratio of 1 peatmoss:2 sterilized Tangi silt loam (from East Feliciana Parish, Louisiana). Seeds were placed on Whatman® filter paper in small glass containers, moistened, covered, and placed in a growth chamber set at 30° C. with 12 hours light per day for 72 hours. Seedlings were then transplanted individually to the soil in benches with 0.20 m sowing distance. Greenhouse temperatures were set to 10-hour day, 29° C. day temperature and 21° C. night temperature. Before the plants flowered, artificial barriers of Tyvek® HomeWrap® (DuPont) tied to 1.2 m bamboo sticks were placed between different EGMS lines for isolation. Individual plants were then harvested, seeds were threshed, cleaned, dried, and weighed at maturity.

In Fall 2020, five promising EGMS lines were selected for seed increase and purification based on the performance of their hybrids in the 2020 TN and Hybrid Preliminary Yield trial. Seeds of EGMS lines 16HT 397-1-6, 16HT 489-1-6, 16HT 695-1-3, 16HT 498-2-4, and 16HT195-2 were directly planted in the soil mix on Oct. 11, 2020 at the greenhouse under the conditions described above for seed production.

### Seed Increase of Male Pollinator Lines

The majority of the pollinator lines were elite inbreds or released varieties showing uniformity in stand and plant type. In Summer 2019, three panicles of each male parent used in 2019 crosses were harvested from the 19XB male nursery, dried, threshed, and stored at 4° C. Male parents of top experimental hybrids based on 2020 yield trials were also harvested. Seeds were threshed, cleaned, weighed after drying, and stored at 4° C.

### Hybrid Seed Production in the Field for Test Cross and Preliminary Yield Trials

Seed production in 2018 field plots was set up in crossing blocks to produce hybrid seed for test cross and preliminary yield trials that used 25 g of seeds per replicate for each hybrid entry. Two-meter rows of EGMS lines planted Mar. 15, 2018 were separated from pollinator lines by a minimum of five meters to reduce pollen contamination. After pollen evaluation, a 100% pollen-sterile female plant that had started heading was planted next a pollinator row/plant that was also heading at the same time. These steps were repeated with other female plants until no space remained next to a pollinator row. PVC pipe frames, 2 m length×1 m width×2 m height, completely covered with Tyvek® HomeWrap® (DuPont) were assembled into isolation boxes to cover the paired rows of males and females and avoid contamination from other male pollen plants. At the time of flowering, supplementary pollination was performed by shaking the male pollen parents with a bamboo stick through an open flap in the isolation box. This process was repeated 3-4 times from 10 AM-1 PM daily during the peak of anthesis for 7-10 days. The same procedure was carried out with a replicate crossing block planted one week after the first crossing block had been established.

To produce more hybrid seeds per combination in 2019, the crossing block set-up was modified into 5-row plots having three rows of each pollinator with two vacant rows for planting selected EGMS female plants at the start of heading. The isolation distance between male plots was 1.5 m, and the Tyvek® isolation boxes were not used in this instance. In 2020, the crossing blocks were further modified into 6-row plots, with two rows of males on each border flanking two vacant rows for planting of selected EGMS plants.

### Hybrid Seed Production for Hybrid Multi-Location Yield Trial (HYBML)

In Summer 2019, seed production was carried out for the three top-yielding hybrids based on the 2018 Test Cross Nursery (TC). These hybrids were 16HT1642-1/PV029 (18TC535), 16HT1380-2/14PVL038-3 (18TC466), and 16HT195-2/14PVL038 (18TC-435).

The set-up was called 19-250, and had a target of producing at least 250 g of seeds for each hybrid cross, the amount normally required per hybrid entry for a multi-location yield trial. Seeds of male and female parents for each cross were planted simultaneously with a Hege 90 planter in 2-meter rows with a 2.5 cm row width. In a six-row plot, four female rows were planted side-by-side, and one row of the male parent flanked the female rows on each side, like border rows. During the booting stage, 2 m high plastic barriers tied to a 1″ PVC pipe were built to cover three sides of each plot. The open side was covered by the barrier of the neighboring plot. Space between the neighboring plots sufficed for cultural management and supplementary pollination. The male-female combinations were planted in duplicate plots when possible, depending on availability of seeds. A total of seven plots were established for the 19-250 seed production of the three hybrids.

Seed production plots (20-250) were also established in Summer 2020 for the four top yielding hybrids from the 2019 TC evaluation. These hybrids were 16HT695-1/PV034 (19TC1-1303), 16HT498-2/RU1702131 (19TC1-1230), 16HT498-2/RU1601010 (19TC1-1238), and 16HT 195-2/PV038-2 (19TC1-1335). Row positions were modified, with male plants in rows 1, 4, and 6, and female plants in rows 2, 3, and 5. The extra row of male plants inside the plots, between female rows, enhanced pollination rates. Three six-row plots per hybrid were planted to produce the desired 250 g of seeds each. Plastic barriers were raised during booting stage every three plots to isolate each hybrid combination. Overall, 12 plots were established for the 20-250 hybrid seed production.

### Results and Discussion

### Seed Increase of EGMS Lines by Ratoon Method

In 2018, a total of 128 plants from 14 EGMS lines ratooned in the field were transferred to the greenhouse for seed production. Grain yield per ratooned plant was 1 to 11 g, with a mean of 1.5 g per plant, equivalent to about 75 seeds per plant. From the 128 plants, 42 plants died or produced under 1 g of seeds. In 2019, 95 plants from 11 distinct EGMS lines were ratooned and grown in the greenhouse and growth chamber. The grain yield was 0 to 7 g, with a mean of 2.1 g per plant (about 105 seeds per ratooned plant). Twelve plants died or produced less than 1 g of seeds.

### Seed Increase of EGMS Lines in the Greenhouse Under Controlled Conditions

A total of 274 plants from six EGMS lines were planted in the greenhouse for seed increase in October 2019. The grain yield was 0 to 33 g, with a mean of 6.0 g per plant (about 300 seeds per plant). Only five plants died or produced under 1 g seeds. EGMS line 16HT695-1-6 produced the most seeds with 458 g, followed by 16HT195-2 (320 g), 16HT695-1-8 (300 g), 16HT498-2-4 (218 g) 16HT695-1-3 (132 g), and 16HT498-2-7 (85 grams).

In October 2020, a total of 780 plants from four EGMS lines were planted in the greenhouse in October 2020 for expected harvest in February 2021.

### Seed Increase of Male Pollinator Lines

All three primary panicles harvested from each pollinator plant in the 2019 male nursery (19XB) were dried and threshed; however, their seed weights were not recorded. All were long grains with ˜150 to 250 grains per panicle. Their seeds were used as seed sources for the 2020XB1 and 2020XB2 male nurseries, as well as for the 20-250 seed production plots. In 2020, two plants of each pollinator line used in the 2020 crosses were harvested from the 20XB1 male nursery. Male parents of the top experimental hybrids, based on the results of the 2020 yield trials, were also harvested. Seed weights per 2 plants are shown in Table 11, ranging from a low of 51 g for Provisia line 14PVL038-1 to a high of 143 g for breeding line RU1802162.

TABLE 11
Seed weight per two plants for pollinator lines used in Summer 2020
crosses, and as male parents of top yielding 2020 experimental hybrids,
H. Rouse Caffey Rice Research Station, Crowley, Louisiana.

| 20XB1_Code | Seed_ID | Seed Wt (g) |
|---|---|---|
| 20XB1-50 | PVL01 | 98 |
| 20XB1-82 | PV027 | 76 |
| 20XB1-85 | RU1601010 | 112 |
| 20XB1-86 | RU1701105 | 96 |
| 20XB1-88 | PVL029 | 93 |
| 20XB1-90 | 14PVL038-1 | 51 |
| 20XB1-91 | 14PVL038-3 | 102 |
| 20XB1-92 | 14PVL038-2 | 75 |
| 20XB1-93 | 18HPD_ID: 4(PVL038) | 107 |
| 20XB1-94 | RU1702131 | 108 |
| 20XB1-95 | RU1703147 | 79 |
| 20XB1-96 | RU1902086 | 98 |
| 20XB1-99 | 17PV001 | 67 |
| 20XB1-100 | Mermentau | 123 |
| 20XB1-101 | RU1802162 | 143 |
| 20XB1-102 | 17PV037 | 96 |
| 20XB1-103 | 17PV027 | 112 |
| 20XB1-104 | 17PV013 | 120 |
| 20XB1-105 | 17PV062 | 81 |
| 20XB1-107 | 17PV 034 | 89 |
| 20XB1-108 | 17PV003 | 98 |
| 20XB1-110 | 17PV006 | 76 |
| 20XB1-111 | 17PV025 | 43 |
| 20XB1-113 | 17PV012 | 99 |
| 20XB1-114 | 17PV024 | 80 |
| 20XB1-116 | Maybelle | 88 |
| 20XB1-117 | PSDO | 72 |
| 20XB1-118 | RU1805198 | 142 |
| 20XB1-119 | Diamond | 102 |
| 20XB1-120 | LKST | 85 |
| 20XB1-121 | RU1902222 | 122 |
| 20XB1-21 | 7252R | 79 |
| 20XB1-23 | R9 | 81 |
| 20XB1-27 | DGL263 | 98 |
| 20XB1-28 | 16R-T270-32 | 84 |
| 20XB1-31 | 17R-T336-52 | 140 |
| 20XB1-32 | 17R-T327-44 | 125 |
| 20XB1-33 | 17R-T324-68 | 117 |
| 20XB1-34 | 17R-T324-44 | 137 |
| 20XB1-35 | 17R-T206-10-2 | 112 |

### Hybrid Seed Production in the Field for Test Cross and Preliminary Yield Trials

The 2018 field crosses produced 120 unique experimental hybrids that were evaluated in the 2019 Test cross Nursery evaluation. Hybrid seed produced per cross was 0 to 50 g with a mean of 6.0 g per plant Nine crosses produced 0 or less than 1 g of seeds, and were not used for further evaluation. Experimental hybrids with more seeds were evaluated in the 2019 test cross in 1 or 2 rows, and some were used in field demonstration plots.

In 2019, 220 crosses were made in the modified field crossing blocks method with 5-row plots. Seed weight per cross was 1 to 32 g, with a mean of 10 g per cross. Only 10 crosses had low seed yields, 1 to 2 g per plant. With higher yields, more than 25 g seed per plant, 37 experimental hybrids were included in the 2020 Hybrid Preliminary Yield trial with a plot area of 8.2 m² per hybrid entry. The 2020 Test cross Nursery was arranged in a Randomized Complete Block Design with 3 single-row replications.

A total of 301 crosses were made in Summer 2020 using a 6-row plot crossing block method with seed weight per cross of 2 to 50 g, and a mean of 10 g.

### Hybrid Seed Production for Hybrid Multi-Location Yield Trial (HYBML)

In the 19-250 hybrid seed production, the highest F₁ seed yield was 184 g from the cross 16HT1642-1/PV029 (18TC-535); followed by 16HT1380-2/14PVL038-3 (18TC-466) with 80 g; and 16HT195-2/14PVL038 (18TC-435) with 8 g of F₁ seeds. These amounts were insufficient for the Hybrid Multi-Location Yield trial, which typically requires 250 g seed. Several problems had been encountered during the conduct of the experiment. One was poor germination of EGMS lines due to cold weather after planting, observed in two plots where 16HT1642-1/PV029 was planted. Also, fertile plants were identified in the 16HT1642 plots. These fertile plants were rogued and discarded, decreasing the number of the EGMS plants. For the two plots with the cross 16HT1380-2/14PVL038-3, the male plants developed about seven days earlier than the female parent. Earlier development of male plants was also observed in the plot with the cross 16HT195-2/14PVL038.

Fertilizer can be used to alter growth rates; urea slows rice growth and development, while muriate of potash hasten development. However, application of fertilizer in this test was largely ineffective for this purpose in the 19-250 runs, because the water was too deep, and it was impractical to drain just the seed production plots without draining the entire field (which also contained many other rice plants involved in various experiments and tests). Gibberellic acid (GA₃) was applied when the plants were at 10% heading at a rate of 100 g/ha for three days. This application rate proved too high for the EGMS lines, which grew as tall as or taller than their male partners after GA₃ treatment.

For the 20-250 hybrid seed production, the set-up was better established and was more productive than the 19-250 seed production. We applied lessons learned from the 19-250 runs. (1) In 2020 the parental seeds were treated with plant growth regulators, fungicides, and insecticides, resulting in better germination and growth. (2) The EGMS parental seeds were easily identified and removed from the plots. (3) There was better synchronization of the parents for three of the four hybrids. Only cross 16HT498-2/RU1601010 (19TC1-1238) had a flowering differential of more than 10 days between male and female parents. (4) GA₃ was applied only twice, at a lower rate of 74 g/ha to address GA₃ sensitivity of the EGMS lines. (5) Supplementary pollination was carried out at the peak of anthesis, every day for 7 to 10 days. Together, these actions and modifications resulted in three hybrid crosses with more than 250 g of F₁ seed yield. Cross 16HT695-1/PV034-2 (19TC1-1303) obtained the highest yield with 2116 g, followed by 16HT498-2/RU1702131 (19TC1-1230) with 782 g, and 16HT195-2/PV038 (19TC1-1335) with 275 g. Thus, these three hybrids were chosen for evaluation in the 2021 Hybrid Multi-Location Yield trial. Seed yield of the cross 16HT498-2/RU1601010 (19TC1-1238) only had 241 g due to poor synchronization.

The production of parental and hybrid seed is an important component of hybrid breeding that is often overlooked. Hybrid yields may be adequate for commercial production, but if parental or hybrid seed production do not meet established thresholds, the corresponding hybrid may not be commercially viable. This study demonstrated that purified EGMS seed can be successfully produced from plants first grown in the field and then transferred for growth chamber treatment and greenhouse seed production. An advantage of this method is that EGMS lines are first selected under field conditions. Relatively small amounts of seed can thereby be reliably produced for initial test cross evaluation. We developed an efficient method for EGMS seed production in the fall greenhouse. This method produced more seed per line, with lower mortality rates than the method previously described for ratooned plants.

These field methods produced sufficient hybrid seed for preliminary and multi-location yield trials. The 6-row seed production plots with three rows of each EGMS and pollinator line produced more than 250 g of seeds for the top three crosses in the 20-250 rows. Planting parents to have approximately the same heading date enhanced seed production. The 6-row seed production method described here is useful to implement a hybrid rice breeding program with limited person-power. Our study identified the preferred practices for successful hybrid seed production in small plots, particularly under but not limited to south Louisiana field conditions: use of pure parent line seeds, seed treatment of parent lines, pairings or plantings based on heading dates of the parents, isolation of seed production plots, GA₃ treatment at 10% heading at a rate of approximately 74 g/ha, and supplementary pollination during the peak of anthesis.

This invention is also directed to methods for producing a rice plant by crossing a first parent rice plant with a second parent rice plant to produce an F₁ hybrid, wherein the first or second rice plant (i.e., female parent) is a rice plant from one of the EGMS lines. Where the parental EGMS line is herbicide-tolerant, the F₁ hybrid preferably displays the herbicide tolerance phenotype of the parental line. Breeding methods that employ the EGMS lines are also part of this invention, including crossing, selfing, backcrossing, hybrid breeding, crossing to populations, the other breeding methods discussed in this specification, and other breeding methods otherwise known to those of skill in the art. Any plants produced using one of the EGMS lines as a parent or ancestor by any of these breeding methods are within the scope of this invention. The other parents or other lines used in such breeding programs may be any of the wide number of rice varieties, cultivars, populations, experimental lines, and other sources of rice germplasm known to the art.

For example, this invention includes methods for producing a first-generation hybrid rice plant by crossing a first parent rice plant with a second parent rice plant, wherein either the first or second parent rice plant. Further, this invention is also directed to methods for producing a hybrid rice line derived by crossing one of the EGMS lines with a second rice plant, and growing the F₁ progeny seed. The crossing and growing steps may be repeated any number of times. Breeding methods using the EGMS rice lines are considered part of this invention, not only backcrossing and hybrid production, but also selfing, crosses to populations, and other breeding methods known in the art. It is preferred that, at each step in the breeding process, there should be selection to maintain the herbicide tolerance trait.

In one embodiment, a rice plant (inbred or hybrid) produced using one of the EGMS lines as a parent or ancestor exhibits tolerance to applications of one or more herbicides or classes of herbicides—including at least the ACCase herbicide tolerance phenotype inherent in one of the herbicide-tolerant EGMS lines; and optionally incorporating one or more additional herbicide tolerance traits as well. The optional, additional herbicide-tolerance trait may be selected from those otherwise known in the art, including those providing tolerance to: acetohydroxyacid synthase (AHAS) inhibitors; bleaching herbicides such as a 4-hydroxyphenylpyruvate dioxygenase (HPPD) inhibitors or phytoene desaturase (PDS) inhibitors; 5-enolpyruvyl shikimate 3-phosphate synthase (EPSPS) inhibitors such as glyphosate; glutamine synthetase (GS) inhibitors such as glufosinate or bialaphos; auxinic herbicides (e.g., an auxin or auxin mimic, an auxin binding protein inhibitor, or auxin transport inhibitor), e.g., dicamba; lipid biosynthesis inhibitors such as ACCase inhibitors; or oxynil (i.e. bromoxynil or ioxynil) herbicides; protoporphyrinogen-IX oxidase (PPO) inhibitors (e.g., acifluorfen, butafenacil, carfentrazone, pyraflufen (e.g., as pyraflufen-ethyl), saflufenacil, trifludimoxazin, flufenpyr-ethyl, fomesafen, flumiclorac, flumioxazin, lactofen, oxadiargyl, oxadiazon, oxyfluorfen, sulfentrazone); lipid biosynthesis inhibitors such as acetyl CoA carboxylase (ACCase) inhibitors; oxynil (i.e. bromoxynil or ioxynil) herbicides; p-hydroxyphenylpyruvate dioxygenase (4-HPPD) inhibitors; amide(s), e.g., propanil; and the like. Examples of AHAS-inhibitor herbicides include, e.g., imidazolinones, sulfonylureas, triazolopyrimidines, pyrimidinyl(thio)benzoates (including pyrimidinyl(oxy)benzoates), sulfonylaminocarbonyltriazolinones, agronomically acceptable salts and esters thereof, and combinations thereof. Examples of ACCase inhibitor herbicides include, e.g., “dims” (e.g., cycloxydim, sethoxydim, clethodim, or tepraloxydim), “fops” (e.g., clodinafop, diclofop, fluazifop, haloxyfop, or quizalofop), and “dens” (such as pinoxaden). Examples of HPPD inhibitors include mesotrione, benzobicyclon, topramezone, tembotrione, and isoxaflutole. Examples of auxinic herbicides include: aminopyralid, dicamba, 2,4-dichlorophenoxyacetic (2,4-D), clopyralid, fluroxypyr, triclopyr or picloram. In addition to dicamba itself, examples of useful dicamba forms include the methyl ester, dimethylamine salt (DMA), diglycoamine salt (DGA), isopropylamine salt (IPA), potassium salt, and sodium salt. In addition to 2,4-D itself, examples of useful 2,4-D forms include the 2-ethylhexyl ester, the iso-octyl ester, the choline salt, the ammonium salt, and the alkylamine salts and alkanolamine salts (specific examples of the latter two including salts with triethylamine (TEA), dimethylamine (DMA), diethylamine, diethanolamine, et al.). Examples of rice plants having imidazolinone-herbicide tolerance are disclosed, for example, in U.S. Pat. Nos. 7,019,196 and 9,090,904.

In some embodiments rice plants that are produced using one of the EGMS lines as a parent or ancestor may be tolerant to ACCase inhibitors, such as the “dims” (e.g., cycloxydim, sethoxydim, clethodim, or tepraloxydim), the “fops” (e.g., clodinafop, diclofop, fluazifop, haloxyfop, or quizalofop), and the “dens” (such as pinoxaden); to auxinic herbicides, such as dicamba; to EPSPS inhibitors, such as glyphosate; to other PPO inhibitors; and to GS inhibitors, such as glufosinate.

In addition to these classes of inhibitors, rice plants that are produced using one of the EGMS lines as a parent or ancestor may also be tolerant to herbicides having other modes of action, for example, chlorophyll/carotenoid pigment inhibitors, cell membrane disruptors, photosynthesis inhibitors, cell division inhibitors, root inhibitors, shoot inhibitors, and combinations thereof.

Such tolerance traits may be expressed, e.g., as mutant acetohydroxyacid synthase large subunit (AHASL) proteins, mutant ACCase proteins, mutant EPSPS proteins, or mutant glutamine synthetase proteins; or as a mutant native, inbred, or transgenic aryloxyalkanoate dioxygenase (AAD or DHT), haloarylnitrilase (BXN), 2,2-dichloropropionic acid dehalogenase (DEH), glyphosate-N-acetyltransferase (GAT), glyphosate decarboxylase (GDC), glyphosate oxidoreductase (GOX), glutathione-S-transferase (GST), phosphinothricin acetyltransferase (PAT or bar), or cytochrome P450 (CYP450) protein having herbicide-degrading activity.

The rice plants hereof can also optionally be “stacked” with other traits including, but not limited to, pesticidal traits such as Bt Cry and other proteins having pesticidal activity toward coleopteran, lepidopteran, nematode, or other pests; nutritional or nutraceutical traits such as modified oil content or oil profile traits, high protein or high amino acid concentration traits, and other trait types known in the art.

Furthermore, in another embodiment, rice plants are generated, e.g. by the use of recombinant DNA techniques, breeding, or otherwise by selection for desired traits, plants that are able to synthesize one or more proteins to improve their productivity, oil content, tolerance to drought, salinity or other growth-limiting environmental factors, or tolerance to arthropod, fungal, bacterial, or viral pests or pathogens of rice plants.

Furthermore, in other embodiments, rice plants are generated, e.g. by the use of recombinant DNA techniques, breeding, or otherwise by selection for desired traits to contain a modified amount of one or more substances or to contain one or more new substances, for example, to improve human or animal nutrition, e.g. health-promoting long-chain omega-3 fatty acids or unsaturated omega-9 fatty acids. (Cf. Nexera® canola, Dow Agro Sciences, Canada).

Furthermore, in some embodiments, rice plants are generated, e.g. by the use of recombinant DNA techniques, breeding, or otherwise by selection for desired traits to contain increased amounts of vitamins, minerals, or improved profiles of nutraceutical compounds.

In one embodiment, rice plants are produced using one of the EGMS lines as a parent or higher-generation ancestor so that the new rice plants, relative to a wild-type rice plant, comprise an increased amount of, or an improved profile of, a compound selected from the group consisting of: glucosinolates (e.g., glucoraphanin (4-methylsulfinylbutyl-glucosinolate), sulforaphane, 3-indolylmethyl-glucosinolate (glucobrassicin), or 1-methoxy-3-indolylmethyl-glucosinolate (neoglucobrassicin)); phenolics (e.g., flavonoids (e.g., quercetin, kaempferol), hydroxycinnamoyl derivatives (e.g., 1,2,2′-trisinapoylgentiobiose, 1,2-diferuloylgentiobiose, 1,2′-disinapoyl-2-feruloylgentiobiose, or 3-O-caffeoyl-quinic (neochlorogenic acid)); and vitamins and minerals (e.g., vitamin C, vitamin E, carotene, folic acid, niacin, riboflavin, thiamine, calcium, iron, magnesium, potassium, selenium, and zinc).

In another embodiment, rice plants are produced using one of the EGMS lines as a parent or higher-generation ancestor so that the new rice plants, relative to a wild-type rice plant, comprise an increased amount of, or an improved profile of, a compound selected from the group consisting of: progoitrin; isothiocyanates; indoles (products of glucosinolate hydrolysis); glutathione; carotenoids such as beta-carotene, lycopene, and the xanthophyll carotenoids such as lutein and zeaxanthin; phenolics comprising the flavonoids such as the flavonols (e.g. quercetin, rutin), the flavins/tannins (such as the procyanidins comprising coumarin, proanthocyanidins, catechins, and anthocyanins); flavones; phytoestrogens such as coumestans; lignans; resveratrol; isoflavones e.g. genistein, daidzein, and glycitein; resorcyclic acid lactones; organosulfur compounds; phytosterols; terpenoids such as carnosol, rosmarinic acid, glycyrrhizin and saponins; chlorophyll; chlorphyllin, sugars, anthocyanins, and vanilla.

### Herbicides

Herbicidal compositions that may be used in conjunction with some aspects of the invention include herbicidally active ingredients (A.I.), and their agronomically acceptable salts and esters. The herbicidal compositions can be applied in any agronomically acceptable format. For example, they can be formulated as ready-to-spray aqueous solutions, powders, or suspensions; as concentrated or highly concentrated aqueous, oily, or other solutions, suspensions, or dispersions; as emulsions, oil dispersions, pastes, dusts, granules, or other broadcastable formats. The herbicidal compositions can be applied by any method known in the art, including, for example, spraying, atomizing, dusting, spreading, watering, seed treatment, or co-planting in admixture with the seed. The particular formulation used depends on the intended purpose; in any case, it should ensure a uniform (or approximately uniform) distribution of the A.I. or A.Is. A herbicidal composition can be selected according to the tolerances of a particular plant, and the plant can be selected from among those having a single tolerance trait, or stacked tolerance traits.

In some embodiments, where the A.I. includes an AHAS inhibitor, the AHAS inhibitor may be selected from: (1) the imidazolinones, e.g. imazamox, imazethapyr, imazapyr, imazapic, imazaquin, and imazamethabenz; preferably imazamox, imazethapyr, imazapyr, or imazapic; (2) the sulfonylureas, e.g. amidosulfuron, azimsulfuron, bensulfuron, cinosulfuron, ethoxysulfuron, flupyrsulfuron, foramsulfuron, imazosulfuron, iodosulfuron, mesosulfuron, metsulfuron, nicosulfuron, thifensulfuron, and tribenuron; (3) the pyrimidinyloxy[thio]benzoates, e.g. including the pyrimidinyloxybenzoates (e.g., bispyribac, pyriminobac, and pyribenzoxim) and the pyrimidinylthiobenzoates (e.g., pyrithiobac and pyriftalid); and (4) the sulfonamides, e.g. including the sulfonylaminocarbonyltriazolinones (e.g., flucarbazone and propoxycarbazone) and the triazolopyrimidines (e.g., cloransulam, diclosulam, florasulam, flumetsulam, metosulam, and penoxsulam). The agronomically acceptable salts and esters of the foregoing are also included, as are combinations thereof.

In embodiments in which the A.I. includes an ACCase inhibitor, the ACCase inhibitor may for example be selected from: aryloxyphenoxypropionate (FOP) herbicides, cyclohexanedione (DIM) herbicides, and phenylpyrazoline (DEN) herbicides, and their agronomically acceptable salts and esters. Examples include: the DIMs, e.g., cycloxydim, sethoxydim, clethodim, or tepraloxydim; the FOPs, e.g., clodinafop, diclofop, fluazifop, haloxyfop, or quizalofop; and the DENs, e.g., pinoxaden, Preferred esters of quizalofop or quizalofop-P include the ethyl and tefuryl esters; and preferred esters of haloxyfop or haloxyfop-P include the methyl and etotyl esters. The agronomically acceptable salts and esters of the foregoing are also included, as are combinations thereof.

Examples of herbicides that are ACCase inhibitors include, but are not limited to, cyclohexanedione herbicides (DIMs, also referred to as: cyclohexene oxime cyclohexanedione oxime; and CHD), aryloxyphenoxy propionate herbicides (also referred to as aryloxyphenoxy propanoate; aryloxyphenoxyalkanoate; oxyphenoxy; APP; AOPP; APA; APPA; FOP), and phenylpyrazole herbicides (also known as DENs; and sometimes referred to under the more general class of phenylpyrazoles such as pinoxaden (e.g., herbicides sold under the trade names Axial and Traxos)). In some methods of controlling weeds or growing herbicide-tolerant plants, at least one herbicide is selected from the group consisting of sethoxydim, cycloxydim, tepraloxydim, haloxyfop, haloxyfop-P or a derivative of one of these herbicides. Table 12 lists examples of herbicides that interfere with ACCase activity.

TABLE 12
Examples of ACCase inhibitors.

| ACCase Inhibitor | Class | Company | Examples of Synonyms and Trade Names |
|---|---|---|---|
| alloxydim | DIM | BASF | Fervin, Kusagard, NP-48Na, BAS 9021H, Carbodimedon, |
| | | | Zizalon |
| butroxydim | DIM | Syngenta | Falcon, ICI-A0500, Butroxydim |
| clethodim | DIM | Valent | Select, Prism, Centurion, RE-45601, Motsa |
| Clodinafop-propargyl | FOP | Syngenta | Discover, Topik, CGA 184 927 |
| clofop | FOP | | Fenofibric Acid, Alopex |
| cloproxydim | FOP | | |
| chlorazifop | FOP | | |
| cycloxydim | DIM | BASF | Focus, Laser, Stratos, BAS 517H |
| cyhalofop-butyl | FOP | Dow | Clincher, XDE 537, DEH 112, Barnstorm |
| diclofop-methyl | FOP | Bayer | Hoegrass, Hoelon, Illoxan, HOE 23408, Dichlorfop, |
| | | | Illoxan |
| fenoxaprop-P-ethyl | FOP | Bayer | Super Whip, Option Super, Exel Super, HOE-46360, |
| | | | Aclaim, Puma S, Fusion |
| fenthiaprop | FOP | | Taifun; Joker |
| fluazifop-P-butyl | FOP | Syngenta | Fusilade, Fusilade 2000, Fusilade DX, ICI-A 0009, ICI-A |
| | | | 0005, SL-236, IH-773B, TF-1169, Fusion |
| haloxyfop-etotyl | FOP | Dow | Gallant, DOWCO 453EE |
| haloxyfop-methyl | FOP | Dow | Verdict, DOWCO 453ME |
| haloxyfop-P-methyl | FOP | Dow | Edge, DE 535 |
| isoxapyrifop | FOP | | |
| Metamifop | FOP | Dongbu | NA |
| pinoxaden | DEN | Syngenta | Axial |
| profoxydim | DIM | BASF | Aura, Tetris, BAS 625H, Clefoxydim |
| propaquizafop | FOP | Syngenta | Agil, Shogun, Ro 17-3664, Correct |
| quizalofop-P-ethyl | FOP | DuPont | Assure, Assure II, DPX-Y6202-3, Targa Super, NC-302, |
| | | | Quizafop |
| quizalofop-P-tefuryl | FOP | Uniroyal | Pantera, UBI C4874 |
| sethoxydim | DIM | BASF | Poast, Poast Plus, NABU, Fervinal, NP-55, Sertin, BAS |
| | | | 562H, Cyethoxydim, Rezult |
| tepraloxydim | DIM | BASF | BAS 620H, Aramo, Caloxydim |
| tralkoxydim | DIM | Syngenta | Achieve, Splendor, ICI-A0604, Tralkoxydime, |
| | | | Tralkoxidym |
| trifop | FOP | | |

Examples of herbicides that are auxinic herbicides include, but are not limited to, those shown in Table 13.

TABLE 13
Examples of Auxinic herbicides.
Classification of Auxinic Herbicides
(HRAC Group ‘O’; WSSA Group ‘4’)

| Subgroup | Member Compound |
|---|---|
| Phenoxy- | Clomeprop |
| carboxylic- | cloprop (“3-CPA”) |
| acid Subgroup | 4-chlorophenoxyacetic acid (“4-CPA”) |
| | 2-(4-chlorophenoxy)propionic acid (“4-CPP”) |
| | 2,4-dichlorophenoxy acetic acid (“2,4-D”) |
| | (3,4-dichlorophenoxy)acetic acid (“3,4-DA”) |
| | 4-(2,4-dichlorophenoxy)butyric acid (“2,4-DB”) |
| | 2-(3,4-dichlorophenoxy)propionic acid (“3,4-DP”) |
| | tris[2-(2,4-dichlorophenoxy)ethyl]phosphite (“2,4-DEP”) |
| | dichlorprop (“2,4-DP”) |
| | 2,4,5-trichlorophenoxyacetic acid (“2,4,5-T”) |
| | fenoprop (“2,4,5-TP”) |
| | 2-(4-chloro-2-methylphenoxy)acetic acid (“MCPA”) |
| | 4-(4-chloro-2-methylphenoxy)butyric acid (“MCPB”) |
| | mecoprop (“MCPP”) |
| Benzoic acid | Chloramben |
| Subgroup | Dicamba |
| | Tricamba |
| | 2,3,6-trichlorobenzoic acid (“TBA”) |
| Pyridine | Aminopyralid |
| carboxylic | Clopyralid |
| acid Subgroup | Fluroxypyr |
| | Picloram |
| | Triclopyr |
| Quinoline | Quinclorac |
| carboxylic | Quinmerac |
| acid Subgroup | |
| Other | Benazolin |
| Subgroup | |

Optional A.I.s of other types include, but are not limited to agronomically-acceptable fungicides such as strobilurins, e.g., pyraclostrobin, alone or in combination with, e.g., boscalid, epiconazole, metaconazole, tebuconazole, kresoxim-methyl, and the like; insecticides, nematicides, lepidoptericides, coleoptericides, or molluscicides (e.g., malathion, pyrethrins/pyrethrum, carbaryl, spinosad, permethrin, bifenthrin, and esfenvalerate).

In one embodiment, a saflufenacil A.I. is, e.g.: 2-chloro-5-[3,6-dihydro-3-methyl-2,6-dioxo-4-(trifluoromethyl)-1-(2H)-pyrimidinyl]-4-fluoro-N-[[methyl(1-methylethyl) amino] sulfonyl]benzamide (CAS: N′-{2-chloro-4-fluoro-5-[1,2,3,6-tetrahydro-3-methyl-2,6-dioxo-4-(trifluoromethyl)pyrimidin-1-yl] benzoyl}-N-isopropyl-N-methylsulfamide; Reg. No.: 372137-35-4); BAS-H800).

As used herein, unless context clearly indicates others, a reference to a named compound, (e.g., “saflufenacil”) should be understood to include not only the specified compound itself, but also the compound's various salts and esters.

The herbicidal compositions can also comprise auxiliary ingredients that are customary for the formulation of crop protection agents. Examples of auxiliaries customary for the formulation of crop protection agents include inert auxiliaries, solid carriers, surfactants (such as dispersants, protective colloids, emulsifiers, wetting agents, and tackifiers), organic and inorganic thickeners, penetrants (such as penetration-enhancing organosilicone surfactants or acidic sulfate chelates, e.g., CT-301™ available from Cheltec, Inc.), safeners, bactericides, antifreeze agents, antifoams, colorants, and adhesives. Formulations of the herbicide compositions useful herein can be prepared according to any method useful for that purpose in the art.

Examples of thickeners (i.e. compounds that modify flow properties, e.g. high viscosity in a state of rest and low viscosity in motion) include polysaccharides, such as xanthan gum (Kelzan® from Kelco), Rhodopol® 23 (Rhone Poulenc) or Veegum® (from R.T. Vanderbilt), and also various organic and inorganic sheet minerals, such as Attaclay® (from Engelhard).

Examples of antifoaming agents include silicone emulsions (for example, Silikon® SRE, Wacker or Rhodorsil® from Rhodia), long-chain alcohols, fatty acids, salts of fatty acids, organofluorine compounds, and mixtures thereof.

Bactericides can optionally be added for stabilizing the aqueous herbicidal formulations. Examples include bactericides based on diclorophen and benzyl alcohol hemiformal (Proxel® from ICI, Acticide® RS from Thor Chemie, or Kathon® MK from Rohm & Haas), or isothiazolinone derivatives, such as alkylisothiazolinones and benzisothiazolinones (Acticide MBS from Thor Chemie).

Examples of antifreeze agents include ethylene glycol, propylene glycol, urea, and glycerol.

Examples of colorants include members of colorant classes such as the sparingly water-soluble pigments and the water-soluble dyes. Some examples include the dyes known under the names Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, and basic red 108.

Examples of adhesives include polyvinylpyrrolidone, polyvinyl acetate, polyvinyl alcohol, and tylose.

Suitable inert auxiliaries include, for example, the following: mineral oil fractions of medium to high boiling point, such as kerosene and diesel oil; coal tar oils; oils of vegetable or animal origin; aliphatic, cyclic and aromatic hydrocarbons, for example paraffins, tetrahydronaphthalene, alkylated naphthalenes and their derivatives, and alkylated benzenes and their derivatives; alcohols such as methanol, ethanol, propanol, butanol and cyclohexanol; ketones such as cyclohexanone; strongly polar solvents, for example amines such as N-methylpyrrolidone; and water; as well as mixtures thereof.

Suitable carriers include liquid and solid carriers.

Liquid carriers include e.g. non-aqueous solvents such as cyclic and aromatic hydrocarbons, e.g. paraffins, tetrahydronaphthalene, alkylated naphthalenes and their derivatives, and alkylated benzenes and their derivatives; alcohols such as methanol, ethanol, propanol, butanol and cyclohexanol; ketones such as cyclohexanone; strongly polar solvents, e.g. amines such as N-methylpyrrolidone; and water; as well as mixtures thereof.

Solid carriers include e.g. mineral earths such as silicas, silica gels, silicates, talc, kaolin, limestone, lime, chalk, bole, loess, clay, dolomite, diatomaceous earth, calcium sulfate, magnesium sulfate, and magnesium oxide; ground synthetic materials; fertilizers such as ammonium sulfate, ammonium phosphate, ammonium nitrate, and ureas; and products of vegetable origin, such as cereal meal, tree bark meal, wood meal, nutshell meal, and cellulose powders; and mixtures thereof.

Suitable surfactants (e.g., adjuvants, wetting agents, tackifiers, dispersants, or emulsifiers) include the alkali metal salts, alkaline earth metal salts, and ammonium salts of aromatic sulfonic acids, for example lignosulfonic acids (e.g. Borrespers-types, Borregaard), phenolsulfonic acids, naphthalenesulfonic acids (Morwet types, Akzo Nobel) and dibutylnaphthalenesulfonic acid (Nekal types, BASF AG); and salts of fatty acids, alkyl- and alkylarylsulfonates, alkyl sulfates, lauryl ether sulfates and fatty alcohol sulfates; and salts of sulfated hexa-, hepta- and octadecanols; fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenol ether, ethoxylated isooctyl-, octyl- or nonylphenol, alkylphenyl or tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters; lignosulfite waste liquors; and proteins, denatured proteins, polysaccharides (e.g. methylcellulose), hydrophobically modified starches, polyvinyl alcohol (Mowiol types, Clariant), polycarboxylates (BASF AG, Sokalan types), polyalkoxylates, polyvinylamine (BASF AG, Lupamine types), polyethyleneimine (BASF AG, Lupasol types), polyvinylpyrrolidone, and copolymers thereof, and mixtures thereof.

Powders, materials for broadcasting and dusts can be prepared by mixing or concomitant grinding of the A.I.s together with a solid carrier.

Granules, for example coated granules, impregnated granules, and homogeneous granules, can be prepared by binding the A.I.s to solid carriers.

Aqueous-use forms can be prepared from emulsion concentrates, suspensions, pastes, wettable powders, or water-dispersible granules by adding water.

To prepare emulsions, pastes, or oil dispersions, the herbicidal compositions can be homogenized in water by means of a wetting agent, tackifier, dispersant or emulsifier. Alternatively, it is also possible to prepare concentrates comprising active compound, wetting agent, tackifier, dispersant or emulsifier and, if desired, solvent or oil, preferably suitable for dilution or dispersion with water.

The concentration of the herbicide(s) present in the herbicidal composition can be varied within wide ranges. In general, the formulations comprise approximately from 0.001% to 98% by weight, preferably 0.01 to 95% by weight of at least one active ingredient. In some embodiments, the A.I.s are employed in a purity of from 90% to 100%, preferably 95% to 100% (as measured, e.g., by NMR or IR spectra).

In some formulations, the herbicides are suspended, emulsified, or dissolved. The formulations may be in the form of aqueous solutions, powders, suspensions, or highly-concentrated aqueous, oily, or other suspensions or dispersions, aqueous emulsions, aqueous microemulsions, aqueous suspo-emulsions, oil dispersions, pastes, dusts, materials for spreading, or granules.

Herbicides or herbicidal compositions can be applied pre-emergence, post-emergence, or pre-planting, or together with the seed. It is also possible to apply the herbicidal composition or active compounds by planting seed pretreated with the herbicidal compositions or active compounds.

In a further embodiment, the herbicides or herbicidal compositions can be applied by treating seed. The treatment of seeds comprises any of the procedures known in the art (e.g., seed dressing, seed coating, seed dusting, seed soaking, seed film coating, seed multilayer coating, seed encrusting, seed dripping, and seed pelleting). The herbicidal compositions can be applied diluted or undiluted.

It may be beneficial in some embodiments to apply the herbicides alone or in combination with other herbicides, or in the form of a mixture with other crop protection agents, for example together with agents for controlling pests or phytopathogenic fungi or bacteria. Also of interest is miscibility with mineral salt solutions, which are employed for treating nutritional and trace element deficiencies. Other additives such as non-phytotoxic oils and oil concentrates can also be added.

Moreover, it may be useful to apply the herbicides in combination with safeners. Safeners are compounds that prevent or reduce herbicide-induced injury to useful plants without having a major effect on the intended herbicidal action of the herbicides. They can be applied either before sowing (e.g. on seed treatments, shoots, or seedlings) or in the pre-emergence application or post-emergence application of the crop plant. The safeners and the herbicides can be applied simultaneously or in succession.

Safeners include e.g. (quinolin-8-oxy)acetic acids, 1-phenyl-5-haloalkyl-1H-1,2,4-triazol-3-carboxylic acids, 1-phenyl-4,5-dihydro-5-alkyl-1H-pyrazol-3,5-dicarboxylic acids, 4,5-dihydro-5,5-diaryl-3-isoxazol carboxylic acids, dichloroacetamides, alpha-oximinophenylacetonitriles, acetophenonoximes, 4,6-dihalo-2-phenylpyrimidines, N-[[4-(aminocarbonyl)phenyl]sulfonyl]-2-benzoic amides, 1,8-naphthalic anhydride, 2-halo-4-(haloalkyl)-5-thiazol carboxylic acids, benoxacor, cloquintocet, cyometrinil, cyprosulfamide, dichlormid, dicyclonon, dietholate, fenchlorazole, fenclorim, flurazole, fluxofenim, furilazole, isoxadifen, mefenpyr, mephenate, naphthalic anhydride, oxabetrinil, 4-(dichloroacetyl)-1-oxa-4-azaspiro[4.5]decane (MON4660, CAS 71526-07-3) and 2,2,5-trimethyl-3-(dichloroacetyl)-1,3-oxazolidine (R-29148, CAS 52836-31-4), phosphorthiolates, and N-alkyl-O-phenyl-carbamates and their agriculturally-acceptable salts and their agriculturally-acceptable derivatives such amides, esters, and thioesters.

Those skilled in the art will recognize that some compounds used as herbicides, safeners, etc. are capable of forming geometric isomers, for example E/Z isomers, enantiomers, diastereomers, or other stereoisomers. In general, it is possible to use either pure isomers or mixtures of isomers. For example, some of the aryloxyphenoxy propionate herbicides are chiral, and some of them are commonly used in enantiomerically enriched or enantiopure form, e.g. clodinafop, cyhalofop, fenoxaprop-P, fluazifop-P, haloxyfop-P, metamifop, propaquizafop or quizalofop-P. As a further example, glufosinate may be used in enantiomerically enriched or enantiopure form, also known as glufosinate-P. Alternatively, the compounds may be used in racemic mixtures or other mixtures of geometric isomers.

### Controlling Weeds

Rice plants of some embodiments of the invention can be used in conjunction with herbicide(s) to which they are tolerant. Herbicides can be applied to the rice plants of the invention using any techniques known to those skilled in the art. Herbicides can be applied at any point in the rice plant cultivation process. For example, herbicides can be applied pre-planting, at planting, pre-emergence, post-emergence, or combinations thereof. Herbicides may be applied to seeds and dried to form a layer on the seeds.

In some embodiments, seeds are treated with a safener, followed by a post-emergence application of herbicide(s). In one embodiment, the post-emergence application of herbicide(s) occurs about 7 to 10 days following planting of safener-treated seeds. In some embodiments, the safener is cloquintocet, dichlormid, fluxofenim, or combinations thereof.

In other aspects, the present invention provides a method for controlling weeds at a locus for growth of a rice plant or plant part thereof, the method comprising applying a composition comprising herbicide(s) to the locus.

In some aspects, the present invention provides a method for controlling weeds at a locus for growth of a plant, the method comprising applying a herbicide composition to the locus; wherein said locus is: (a) a locus that contains a rice plant or seed capable of producing a rice plant; or (b) a locus that will contain the rice plant or the seed after the herbicide composition is applied.

Following are non-limiting examples of various rice culturing methods, including the application of herbicide(s).

In the post-flood, post-emergence (transplanted) method, rice is grown to about the 2-4 leaf stage away from the field. The field is flooded and tilled (puddled) until a blend of mud is achieved. The rice plants are then transplanted into the mud. Herbicide application typically takes place before or after flooding.

In the post-flood, post-emergence (water-seeded) method, rice is soaked for about 24 hours or more, and then is sown into the surface of a shallow flooded field. Herbicide application is typically made after weed germination.

In the pre-flood, post-emergence, direct-seeded (broadcast or drilled) method, rice is broadcast or planted with a planter under the soil surface. The field may be flushed (watered) to promote rice growth. The field is flooded about a week or more after planting as the plants germinate. Herbicide application takes place typically before the flood, but after emergence of the rice plants.

In the pre-flood, post-emergence (Southeast Asia style) method, rice is soaked for about 24 hours or more. The field is puddled to the right consistency and drained. The pre-germinated seeds are then broadcast to the surface of the soil. Flooding takes place as the rice develops. Herbicide application normally takes place before the flooding, but after the emergence of the rice plants.

In the pre-emergence or delayed pre-emergence method, seeds are planted, usually with a planter. Herbicide is applied before emergence of the rice or weeds.

Herbicide compositions can be applied, e.g., as foliar treatments, soil treatments, seed treatments, or soil drenches. Application can be made, e.g., by spraying, dusting, broadcasting, or any other mode known in the art.

In one embodiment, herbicides can be used to control the growth of weeds in the vicinity of the rice plants of the invention. A herbicide to which the rice plant of the invention is tolerant can be applied to the plot at a concentration sufficient to kill or inhibit the growth of weeds. Concentrations of herbicide sufficient to kill or inhibit the growth of weeds are known in the art for typical circumstances, and generally depend on the particulars of the herbicide, the weeds being controlled, the weather, the soil type, the degree of maturity of the weeds, and the like.

In another embodiment, the present invention provides a method for controlling weeds in the vicinity of rice plants. The method comprises applying an effective amount of herbicide(s) to the weeds and to the rice plant, wherein the rice plant has increased tolerance to the herbicide(s) when compared to a wild-type rice plant. An “effective amount” of herbicide is an amount that is sufficient to kill or inhibit the growth of particular weeds. What constitutes an “effective amount” depends on the particulars of the herbicide, the weeds being controlled, the weather, the soil type, the degree of maturity of the weeds, and the like; such “effective amounts” for typical circumstances are well known in the art.

In another aspect, herbicide(s) can be used as a seed treatment. In some embodiments, an effective concentration or an effective amount of herbicide(s), or a composition comprising an effective concentration or an effective amount of herbicide(s) can be applied directly to the seeds prior to or during the sowing of the seeds. Seed treatment formulations may additionally comprise binders, and optionally colorants as well.

Binders can be added to improve the adhesion of the active materials onto the seeds after treatment. Suitable binders include, e.g., block copolymers, EO/PO surfactants, polyvinylalcohols, polyvinylpyrrolidones, polyacrylates, polymethacrylates, polybutenes, polyisobutylenes, polystyrene, polyethyleneamines, polyethyleneamides, polyethyleneimines (e.g., Lupasol®, Polymin®), polyethers, polyurethanes, polyvinylacetate, tylose, and copolymers derived from these polymers.

The term “seed treatment” includes all suitable seed treatment techniques known in the art, including seed dressing, seed coating, seed dusting, seed soaking, and seed pelleting. Alternatively, or in addition, soil may be treated by applying a formulation containing the herbicide (e.g., a granular formulation), for example with a seed drill, with optionally one or more solid or liquid, agriculturally acceptable carriers, and optionally with one or more agriculturally acceptable surfactants.

The present invention also comprises seeds coated with or containing a seed treatment formulation comprising herbicide(s). The term “coated with or containing” generally signifies that the active ingredient is for the most part on the surface of the seed at the time of application, although a greater or lesser part of the ingredient may penetrate into the seed, depending on the method of application. When the seed is planted, it may absorb the active ingredient.

In some embodiments, the seed treatment with herbicide(s) or with a formulation comprising the herbicide(s) is applied by spraying or dusting the seeds, or otherwise treating the seeds, before the seeds are sown.

In other aspects, the present invention provides a method for combating undesired vegetation or controlling weeds, comprising contacting seeds of the rice plants with herbicide(s) before sowing, or after pre-germination, or both. The method can further comprise sowing the seeds, for example, in soil in a field or in a potting medium in a greenhouse. The method finds particular use in combating undesired vegetation or controlling weeds in the immediate vicinity of the seed. The control of undesired vegetation is understood as the killing of weeds, or otherwise retarding or inhibiting the normal growth of weeds. “Weeds,” in the broadest sense, should be understood as including all plants that grow in locations where they are undesired.

The weeds that may be treated include, for example, dicotyledonous and monocotyledonous weeds. Monocotyledonous weeds include, but are not limited to, weeds of the genera: Echinochloa, Setaria, Panicum, Digitaria, Phleum, Poa, Festuca, Eleusine, Brachiaria, Lolium, Bromus, Avena, Oryza, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, Centrosaurus, and Apera. Dicotyledonous weeds include, but are not limited to, weeds of the genera: Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, Canadaspis, and Taraxacum.

Examples of red/weedy rice include, but are not limited to, Oryza longistaminata, Oryza sativa L. var sylvatica, Oryza latifolia, Oryza barthii A. Chev, Oryza punctata, and Oryza rufipogon.

Examples of Echinochloa spp. include, but are not limited to, Echinochloa colona, Echinochloa crusgalli, and Echinochloa oryzicola.

In addition, the weeds treated with the present invention can include, for example, crop plants that are growing in an undesired location.

In still further aspects, loci, plants, plant parts, or seeds are treated with an agronomically acceptable composition that does not contain an A.I. For example, the treatment may comprise one or more agronomically-acceptable carriers, diluents, excipients, plant growth regulators, and the like; or an adjuvant, such as a surfactant, a spreader, a sticker, a penetrant, a drift-control agent, a crop oil, an emulsifier, a compatibility agent, or combinations thereof.

In other aspects, the present invention provides a product prepared from the rice plants of the invention, for example, brown rice (e.g., cargo rice), broken rice (e.g., chits, brewer's rice), polished rice (e.g., milled rice), rice hulls (e.g., husks, chaff), rice bran, rice pollards, rice mill feed, rice flour, rice oil, oiled rice bran, de-oiled rice bran, arrak, rice wine, poultry litter, and animal feed.

### Further Embodiments of the Invention

As used herein, the term “plant” includes plant cells, plant protoplasts, plant cells of tissue culture from which rice plants can be regenerated, plant calli, plant clumps, and plant cells that are intact in plants or parts of plants, such as pollen, flowers, embryos, ovules, seeds, pods, leaves, stems, roots, anthers, and the like. Thus, another aspect of this invention is to provide for cells that, upon growth and differentiation, produce a cultivar having essentially all of the physiological and morphological characteristics of one of the EGMS lines.

Techniques for transforming with and expressing desired structural genes and cultured cells are known in the art. Also, as known in the art, rice may be transformed and regenerated such that whole plants containing and expressing desired genes under regulatory control are obtained. General descriptions of plant expression vectors and reporter genes and transformation protocols can be found, for example, in Gruber et al., “Vectors for Plant Transformation, in Methods in Plant Molecular Biology & Biotechnology” in Glich et al. (Eds. pp. 89-119, CRC Press, 1993). For example, expression vectors and gene cassettes with the GUS reporter are available from Clone Tech Laboratories, Inc. (Palo Alto, Calif.), and expression vectors and gene cassettes with luciferase reporter are available from Promega Corp. (Madison, Wis.). General methods of culturing plant tissues are provided, for example, by Maki et al., “Procedures for Introducing Foreign DNA into Plants” in Methods in Plant Molecular Biology & Biotechnology, Glich et al., (Eds. pp. 67-88 CRC Press, 1993); by Phillips et al., “Cell-Tissue Culture and In-Vitro Manipulation” in Corn & Corn Improvement, 3rd Edition; and by Sprague et al., (Eds. pp. 345-387) American Society of Agronomy Inc., 1988. Methods of introducing expression vectors into plant tissue include the direct infection or co-cultivation of plant cells with Agrobacterium tumefaciens, Horsch et al., Science, 227:1229 (1985). Descriptions of Agrobacterium vectors systems and methods for Agrobacterium-mediated gene transfer are provided by Gruber et al., supra.

Useful methods include but are not limited to expression vectors introduced into plant tissues using a direct gene transfer method such as microprojectile-mediated delivery, DNA injection, electroporation, and the like. More preferably expression vectors are introduced into plant tissues using the microprojectile media delivery with biolistic device- or Agrobacterium-mediated transformation. Transformed plants obtained with the germplasm of the disclosed lines are intended to be within the scope of this invention.

The present invention also provides rice plants regenerated from a tissue culture of the disclosed lines or hybrid plant. As is known in the art, tissue culture can be used for the in vitro regeneration of a rice plant. For example, see Chu, Q. R. et al. (1999) “Use of bridging parents with high anther culturability to improve plant regeneration and breeding value in rice,” Rice Biotechnology Quarterly, 38:25-26; Chu, Q. R. et al., “A novel plant regeneration medium for rice anther culture of Southern U.S. crosses,” Rice Biotechnology Quarterly, 35:15-16 (1998); Chu, Q. R. et al., “A novel basal medium for embryogenic callus induction of Southern US crosses,” Rice Biotechnology Quarterly, 32:19-20 (1997); and Oono, K., “Broadening the Genetic Variability By Tissue Culture Methods,” Jap. J. Breed., 33 (Supp. 2), 306-307 (1983). Thus, another aspect of this invention is to provide cells that, upon growth and differentiation, produce rice plants having all, or essentially all, of the physiological and morphological characteristics of one of the EGMS lines.

Unless context clearly indicates otherwise, references in the specification and claims to one of the EGMS lines should be understood also to include single gene conversions of one of those lines with a gene encoding a trait such as, for example, male sterility, other sources of herbicide resistance, resistance for bacterial, fungal, or viral disease, insect resistance, male fertility, enhanced nutritional quality, industrial usage, yield stability and yield enhancement.

Duncan et al., Planta, 165:322-332 (1985) reflects that 97% of the plants cultured that produced callus were capable of plant regeneration. Subsequent experiments with both inbreds and hybrids produced 91% regenerable callus that produced plants. In a further study, Songstad et al., Plant Cell Reports, 7:262-265 (1988) reported several media additions that enhanced regenerability of callus of two inbred lines. Other published reports also indicate that “nontraditional” tissues are capable of producing somatic embryogenesis and plant regeneration. K. P. Rao et al., Maize Genetics Cooperation Newsletter, 60:64-65 (1986), refers to somatic embryogenesis from glume callus cultures and B. V Conger et al., Plant Cell Reports, 6:345-347 (1987) reported somatic embryogenesis from the tissue cultures of corn leaf segments. These methods of obtaining plants are routinely used with a high rate of success.

Tissue culture of corn (maize) is described in European Patent Application No. 160,390. Corn tissue culture procedures, which may be adapted for use with rice, are also described in Green et al., “Plant Regeneration in Tissue Culture of Maize,” Maize for Biological Research (Plant Molecular Biology Association, Charlottesville, Va., pp. 367-372, 1982) and in Duncan et al., “The Production of Callus Capable of Plant Regeneration from Immature Embryos of Numerous Zea Mays Genotypes,” 165 Planta, 322:332 (1985). Thus, another aspect of this invention is to provide cells that, upon growth and differentiation, produce rice plants having all, or essentially all, of the physiological and morphological characteristics of one of the disclosed lines or their hybrids. See T. P. Croughan et al., (Springer-Verlag, Berlin, 1991) Rice (Oryza sativa L.): Establishment of Callus Culture and the regeneration of Plants, in Biotechnology in Agriculture and Forestry (19-37).

With the advent of molecular biological techniques that allow the isolation and characterization of genes that encode specific protein products, it is now possible to routinely engineer plant genomes to incorporate and express foreign genes, or additional or modified versions of native, or endogenous, genes (perhaps driven by different promoters) in order to alter the traits of a plant in a specific manner. Such foreign, additional, and modified genes are herein referred to collectively as “transgenes.” In recent years, several methods for producing transgenic plants have been developed, and the present invention, in particular embodiments, also relates to transformed versions of the disclosed lines.

An expression vector is constructed that will function in plant cells. Such a vector comprises a DNA coding sequence that is under the control of or is operatively linked to a regulatory element (e.g., a promoter). The expression vector may contain one or more such operably linked coding sequence/regulatory element combinations. The vector(s) may be in the form of a plasmid or virus, and can be used alone or in combination with other plasmids or viruses to provide transformed rice plants.

### Expression Vectors

Expression vectors commonly include at least one genetic “marker,” operably linked to a regulatory element (e.g., a promoter) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are known in the art, and include, for example, genes that code for enzymes that metabolically detoxify a selective chemical inhibitor such as an antibiotic or a herbicide, or genes that encode an altered target that is insensitive to such an inhibitor. Positive selection methods are also known in the art.

For example, a commonly used selectable marker gene for plant transformation is that for neomycin phosphotransferase II (nptII), isolated from transposon Tn5, whose expression confers resistance to kanamycin. See Fraley et al., Proc. Natl. Acad. Sci. U.S.A., 80:4803 (1983). Another commonly used selectable marker gene is the hygromycin phosphotransferase gene, which confers resistance to the antibiotic hygromycin. See Vanden Elzen et al., Plant Mol. Biol., 5:299 (1985).

Additional selectable marker genes of bacterial origin that confer resistance to one or more antibiotics include gentamycin acetyl transferase, streptomycin phosphotransferase, aminoglycoside-3′-adenyl transferase, and the bleomycin resistance determinant. Hayford et al., Plant Physiol., 86:1216 (1988), Jones et al., Mol. Gen. Genet., 210:86 (1987), Svab et al., Plant Mol. Biol., 14:197 (1990); Plant Mol. Biol., 7:171 (1986). Other selectable marker genes confer resistance to herbicides such as glyphosate, glufosinate, or broxynil. Comai et al., Nature, 317:741-744 (1985); Gordon-Kamm et al., Plant Cell, 2:603-618 (1990); and Stalker et al., Science, 242:419-423 (1988).

Selectable marker genes for plant transformation of non-bacterial origin include, for example, mouse dihydrofolate reductase, plant 5-enolpyruvylshikimate-3-phosphate synthase, and plant acetolactate synthase. Eichholtz et al., Somatic Cell Mol. Genet. 13:67 (1987); Shah et al., Science, 233:478 (1986); and Charest et al., Plant Cell Rep., 8:643 (1990).

Another class of marker genes for plant transformation employs screening of presumptively transformed plant cells, rather than selection for resistance to a toxic substance such as an antibiotic. These marker genes are particularly useful to quantify or visualize the spatial pattern of expression of a gene in specific tissues, and are frequently referred to as reporter genes because they may be fused to the target gene or regulatory sequence. Commonly used reporter genes include glucuronidase (GUS), galactosidase, luciferase, chloramphenicol, and acetyltransferase. See Jefferson, R. A., Plant Mol. Biol. Rep., 5:387 (1987); Teeri et al., EMBO J., 8:343 (1989); Koncz et al., Proc. Natl. Acad. Sci. U.S.A., 84:131 (1987); and DeBlock et al., EMBO J., 3:1681 (1984). Another approach to identifying relatively rare transformation events has been the use of a gene that encodes a dominant constitutive regulator of the Zea mays anthocyanin pigmentation pathway. Ludwig et al., Science, 247:449 (1990).

The Green Fluorescent Protein (GFP) gene has been used as a marker for gene expression in prokaryotic and eukaryotic cells. See Chalfie et al., Science, 263:802 (1994). GFP and mutants of GFP may be used as screenable markers.

Genes included in expression vectors are driven by a nucleotide sequence comprising a regulatory element, for example, a promoter. Many suitable promoters are known in the art, as are other regulatory elements that may be used either alone or in combination with promoters.

As used herein, “promoter” refers to a region of DNA upstream or downstream from the transcription initiation site, a region that is involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred.” Promoters that initiate transcription only in certain tissue are referred to as “tissue-specific.” A “cell type” specific promoter primarily drives expression in certain cell types in one or more organs, for example, vascular cells in roots or leaves. An “inducible” promoter is a promoter that is under environmental control. Examples of environmental conditions that may induce transcription by inducible promoters include anaerobic conditions or the presence of light. Tissue-specific, tissue-preferred, cell type specific, and inducible promoters are examples of “non-constitutive” promoters. A “constitutive” promoter is a promoter that is generally active under most environmental conditions.

### A. Inducible Promoters

An inducible promoter is operably linked to a gene for expression in rice. Optionally, the inducible promoter is operably linked to a nucleotide sequence encoding a signal sequence that is operably linked to a gene for expression in rice. With an inducible promoter the rate of transcription increases in response to an inducing agent.

Any suitable inducible promoter may be used in the present invention. See Ward et al., Plant Mol. Biol., 22:361-366 (1993). Examples include those from the ACEI system, which responds to copper, Meft et al., PNAS, 90:4567-4571 (1993); In2 gene from maize, which responds to benzenesulfonamide herbicide safeners, Hershey et al., Mol. Gen Genetics, 227:229-237 (1991); Gatz et al., Mol. Gen. Genetics, 243:32-38 (1994); and Tet repressor from Tn10, Gatz, Mol. Gen. Genetics, 227:229-237 (1991). A preferred inducible promoter is one that responds to an inducing agent to which plants do not normally respond, for example, the inducible promoter from a steroid hormone gene, the transcriptional activity of which is induced by a glucocorticosteroid hormone. See Schena et al., Proc. Natl. Acad. Sci., U.S.A. 88:0421 (1991).

### B. Constitutive Promoters

A constitutive promoter is operably linked to a gene for expression in rice, or the constitutive promoter is operably linked to a nucleotide sequence encoding a signal sequence that is operably linked to a gene for expression in rice.

Constitutive promoters may also be used in the instant invention. Examples include promoters from plant viruses such as the 35S promoter from cauliflower mosaic virus, Odell et al., Nature, 313:810-812 (1985), and the promoters from the rice actin gene, McElroy et al., Plant Cell, 2:163-171 (1990); ubiquitin, Christensen et al., Plant Mol. Biol., 12:619-632 (1989) and Christensen et al., Plant Mol. Biol. 18:675-689 (1992); pEMU, Last et al., Theor. Appl. Genet., 81:581-588 (1991); MAS, Velten et al., EMBO J., 3:2723-2730 (1984); and maize H3 histone, Lepetit et al., Mol. Gen. Genetics, 231:276-285 (1992) and Atanassova et al., Plant Journal, 2 (3): 291-300 (1992). An ACCase promoter, such as a rice ACCase promoter, may be used as a constitutive promoter.

### C. Tissue-Specific or Tissue-Preferred Promoters

A tissue-specific promoter is operably linked to a gene for expression in rice. Optionally, the tissue-specific promoter is operably linked to a nucleotide sequence encoding a signal sequence that is operably linked to a gene for expression in rice. Transformed plants produce the expression product of the transgene exclusively, or preferentially, in specific tissue(s).

Any tissue-specific or tissue-preferred promoter may be used in the instant invention. Examples of tissue-specific or tissue-preferred promoters include those from the phaseolin gene, Murai et al., Science, 23:476-482 (1983), and Sengupta-Gopalan et al., Proc. Natl. Acad. Sci. U.S.A., 82:3320-3324 (1985); a leaf-specific and light-induced promoter such as that from cab or rubisco, Simpson et al., EMBO J., 4(11):2723-2729 (1985) and Timko et al., Nature, 318:579-582 (1985); an anther-specific promoter such as that from LAT52, Twell et al., Mol. Gen. Genetics, 217:240-245 (1989); a pollen-specific promoter such as that from Zm13, Guerrero et al., Mol. Gen. Genetics, 244:161-168 (1993); or a microspore-preferred promoter such as that from apg, Twell et al., Sex. Plant Reprod., 6:217-224 (1993).

### Signal Sequences for Targeting Proteins to Subcellular Compartments

Transport of protein or peptide molecules produced by transgenes to a subcellular compartment such as a chloroplast, vacuole, peroxisome, glyoxysome, cell wall, or mitochondrion, or for secretion into an apoplast, is accomplished by operably linking a nucleotide sequence encoding a signal sequence to the 5′ or 3′ end of a gene encoding the protein or peptide of interest. Targeting sequences at the 5′ or 3′ end of the structural gene may determine, during protein synthesis and processing, where the encoded protein is ultimately compartmentalized.

Many signal sequences are known in the art. See, for example, Becker et al., Plant Mol. Biol., 20:49 (1992); Close, P. S., Master's Thesis, Iowa State University (1993); Knox, C. et al., “Structure and Organization of Two Divergent Alpha-Amylase Genes from Barley,” Plant Mol. Biol., 9:3-17 (1987); Lerner et al., Plant Physiol., 91:124-129 (1989); Fontes et al., Plant Cell, 3:483-496 (1991); Matsuoka et al., Proc. Natl. Acad. Sci., 88:834 (1991); Gould et al., J. Cell. Biol., 108:1657 (1989); Creissen et al., Plant J., 2:129 (1991); Kalderon et al., “A short amino acid sequence able to specify nuclear location,” Cell, 39:499-509 (1984); and Steifel et al., “Expression of a maize cell wall hydroxyproline-rich glycoprotein gene in early leaf and root vascular differentiation,” Plant Cell, 2:785-793 (1990).

### Foreign Protein Genes and Agronomic Genes

Agronomically significant genes that may be transformed into rice plants in accordance with the present invention include, for example, the following:

1. Genes that Confer Resistance to Pests or Disease:
A. Plant disease resistance genes. Plant defenses are often activated by specific interaction between the product of a disease resistance gene (R) in the plant and the product of a corresponding avirulence (Avr) gene in the pathogen. A plant may be transformed with a cloned resistance gene to engineer plants that are resistant to specific pathogen strains. See, e.g., Jones et al., Science 266:789 (1994) (cloning of the tomato Cf-9 gene for resistance to Cladosporium fulvum); Martin et al., Science 262:1432 (1993) (tomato Pto gene for resistance to Pseudomonas syringae pv. Tomato encodes a protein kinase); and Mindrinos et al., Cell 78:1089 (1994) (Arabidopsis RSP2 gene for resistance to Pseudomonas syringae).
B. A Bacillus thuringiensis protein, a derivative thereof, or a synthetic polypeptide modeled thereon. See, e.g., Geiser et al., Gene 48:109 (1986), disclosing the cloning and nucleotide sequence of a Bt-endotoxin gene. DNA molecules encoding endotoxin genes may be obtained from American Type Culture Collection, Manassas, Va., e.g., under ATCC Accession Nos. 40098, 67136, 31995, and 31998.
C. A lectin. See, for example, Van Damme et al., Plant Molec. Biol. 24:25 (1994), disclosing the nucleotide sequences of several Clivia miniata mannose-binding lectin genes.
D. A vitamin-binding protein such as avidin. See PCT Application US93/06487. This disclosure teaches the use of avidin and avidin homologues as larvicides against insect pests.
E. An enzyme inhibitor, e.g., a protease or proteinase inhibitor or an amylase inhibitor. See, e.g., Abe et al., J. Biol. Chem. 262:16793 (1987) (nucleotide sequence of rice cysteine proteinase inhibitor); Huub et al., Plant Molec. Biol. 21:985 (1993) (nucleotide sequence of cDNA encoding tobacco proteinase inhibitor 1); and Sumitani et al., Biosci. Biotech. Biochem. 57:1243 (1993) (nucleotide sequence of Streptomyces nitrosporeus-amylase inhibitor).
F. An insect-specific hormone or pheromone such as an ecdysteroid and juvenile hormone, a variant thereof, a mimetic based thereon, or an antagonist or agonist thereof. See, e.g., Hammock et al., Nature, 344:458 (1990), disclosing baculovirus expression of cloned juvenile hormone esterase, an inactivator of juvenile hormone.
G. An insect-specific peptide or neuropeptide that, upon expression, disrupts the physiology of the affected pest. See, e.g., Regan, J. Biol. Chem. 269:9 (1994) (expression cloning yields DNA coding for insect diuretic hormone receptor); and Pratt et al., Biochem. Biophys. Res. Comm., 163:1243 (1989) (an allostatin in Diploptera puntata). See also U.S. Pat. No. 5,266,317 to Tomalski et al., disclosing genes encoding insect-specific, paralytic neurotoxins.
H. An insect-specific venom produced in nature by a snake, a wasp, etc. For example, see Pang et al., Gene, 116:165 (1992), concerning heterologous expression in plants of a gene coding for a scorpion insectotoxic peptide.
I. An enzyme responsible for hyperaccumulation of a monoterpene, a sesquiterpene, a steroid, hydroxamic acid, a phenylpropanoid derivative or another non-protein molecule with insecticidal activity.
J. An enzyme involved in the modification, including post-translational modification, of a biologically active molecule; e.g., a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase, or a glucanase, either natural or synthetic. See PCT Application WO 9302197 to Scott et al., which discloses the nucleotide sequence of a callase gene. DNA molecules that contain chitinase-encoding sequences can be obtained, for example, from the American Type Culture Collection under Accession Nos. 39637 and 67152. See also Kramer et al., Insect Biochem. Molec. Biol. 23:691 (1993), which discloses the nucleotide sequence of a cDNA encoding tobacco hookworm chitinase; and Kawalleck et al., Plant Molec. Biol., 21:673 (1993), which discloses the nucleotide sequence of the parsley ubi4-2 polyubiquitin gene.
K. A molecule that stimulates signal transduction. See, e.g., Botella et al., Plant Molec. Biol., 24:757 (1994), which discloses nucleotide sequences for mung bean calmodulin cDNA clones; and Griess et al., Plant Physiol., 104:1467 (1994), which discloses the nucleotide sequence of a maize calmodulin cDNA clone.
L. An antimicrobial or amphipathic peptide. See PCT Application WO 9516776 (disclosing peptide derivatives of Tachyplesin that inhibit fungal plant pathogens); and PCT Application WO 9518855 (disclosing synthetic antimicrobial peptides that confer disease resistance).
M. A membrane permease, a channel former or a channel blocker. See, e.g., Jaynes et al., Plant Sci., 89:43 (1993), which discloses heterologous expression of a cecropin lytic peptide analog to render transgenic tobacco plants resistant to Pseudomonas solanacearum.
N. A viral-invasive protein or a complex toxin derived therefrom. For example, the accumulation of viral coat proteins in transformed plant cells induces resistance to viral infection or disease development caused by the virus from which the coat protein gene is derived, as well as by related viruses. Coat protein-mediated resistance has been conferred upon transformed plants against alfalfa mosaic virus, cucumber mosaic virus, tobacco streak virus, potato virus X, potato virus Y, tobacco etch virus, tobacco rattle virus, and tobacco mosaic virus. See Beachy et al., Ann. Rev. Phytopathol., 28:451 (1990).
O. An insect-specific antibody or an immunotoxin derived therefrom. Thus, an antibody targeted to a critical metabolic function in the insect gut inactivates an affected enzyme, killing the insect. See Taylor et al., Abstract #497, Seventh Int'l Symposium on Molecular Plant-Microbe Interactions (Edinburgh, Scotland, 1994) (enzymatic inactivation in transgenic tobacco via production of single-chain antibody fragments).
P. A virus-specific antibody. See, e.g., Tavladoraki et al., Nature, 366:469 (1993), showing protection of transgenic plants expressing recombinant antibody genes from virus attack.
Q. A developmental-arrest protein produced in nature by a pathogen or a parasite. For example, fungal endo-1,4-D-polygalacturonases facilitate fungal colonization and plant nutrient release by solubilizing plant cell wall homo-1,4-D-galacturonase. See Lamb et al., Bio/Technology, 10:1436 (1992). The cloning and characterization of a gene that encodes a bean endopolygalacturonase-inhibiting protein is described by Toubart et al., Plant J., 2:367 (1992).
R. A developmental-arrest protein produced in nature by a plant. For example, Logemann et al., Bio/Technology, 10:305 (1992) reported that transgenic plants expressing the barley ribosome-inactivating gene have an increased resistance to fungal disease.
   2. Genes that Confer Additional Resistance to a Herbicide, Beyond that which is Inherent in Certain of the EGMS Lines for Example:
A. A herbicide that inhibits the growing point or meristem, such as an imidazolinone or a sulfonylurea. Exemplary genes in this category code for mutant ALS and AHAS enzymes as described, for example, by Lee et al., EMBO J., 7:1241 (1988); and Miki et al., Theor. Appl. Genet., 80:449 (1990), respectively. See, additionally, U.S. Pat. Nos. 5,545,822; 5,736,629; 5,773,703; 5,773,704; 5,952,553; 6,274,796; 6,943,280; 7,019,196; 7,345,221; 7,399,905; 7,495,153; 7,754,947; 7,786,360; 8,841,525; 8,841,526; 8,946,528; 9,029,642; 9,090,904; and 9,220,220. Resistance to AHAS-acting herbicides may be through a mechanism other than a resistant AHAS enzyme. See, e.g., U.S. Pat. No. 5,545,822.
B. Glyphosate: Resistance may be imparted by mutant 5-enolpyruvl-3-phosphikimate synthase (EPSP) and aroA genes. Other phosphono compounds such as glufosinate: Resistance may be imparted by phosphinothricin acetyl transferase, PAT, and Streptomyces hygroscopicus phosphinothricin-acetyl transferase, bar, genes. Pyridinoxy or phenoxy propionic acids and cyclohexones: Resistance may be imparted by ACCase inhibitor-encoding genes. See, e.g., U.S. Pat. No. 4,940,835 to Shah et al., which discloses the nucleotide sequence of a form of EPSP that confers glyphosate resistance. A DNA molecule encoding a mutant aroA gene can be obtained under ATCC Accession Number 39256, and the nucleotide sequence of the mutant gene is disclosed in U.S. Pat. No. 4,769,061 to Comai. European Patent Application No. 0333033 to Kumada et al.; and U.S. Pat. No. 4,975,374 to Goodman et al., disclose nucleotide sequences of glutamine synthetase genes that confer resistance to herbicides such as L-phosphinothricin. The nucleotide sequence of a phosphinothricin-acetyl-transferase gene is provided in European Application No. 0242246 to Leemans et al. and DeGreef et al., Bio/Technology, 7:61 (1989), describing the production of transgenic plants that express chimeric bar genes coding for phosphinothricin acetyl transferase activity. Examples of genes conferring resistance to phenoxy propionic acids and cyclohexones, such as sethoxydim and haloxyfop, are the Acc1-S1, Acc1-S2, and Acc1-S3 genes described by Marshall et al., Theor. Appl. Genet., 83:435 (1992).
C. A herbicide that inhibits photosynthesis, such as a triazine (psbA and gs+ genes) or a benzonitrile (nitrilase gene). Przibilla et al., Plant Cell, 3:169 (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441, and 67442. Cloning and expression of DNA coding for a glutathione S-transferase is described by Hayes et al., Biochem. J., 285:173 (1992).
   3. Genes that Confer or Contribute to a Value-added Trait, such as:
A. Modified fatty acid metabolism, for example, by transforming a plant with an antisense sequence to stearyl-ACP desaturase, to increase stearic acid content of the plant. See Knultzon et al., Proc. Natl. Acad. Sci. U.S.A. 89:2624 (1992).
B. Decreased Phytate Content
   1) Introduction of a phytase-encoding gene would enhance breakdown of phytate, adding more free phosphate to the transformed plant. See, e.g., Van Hartingsveldt et al., Gene, 127:87 (1993), which discloses the nucleotide sequence of an Aspergillus niger phytase gene.
   2) A gene may be introduced to reduce phytate content. For example, this may be accomplished by cloning, and then reintroducing DNA associated with an allele that is responsible for maize mutants characterized by low levels of phytic acid, or a homologous or analogous mutation in rice may be used. See Raboy et al., Maydica, 35:383 (1990).
C. Carbohydrate composition may be modified, for example, by transforming plants with a gene coding for an enzyme that alters the branching pattern of starch. See Shiroza et al., J. Bacteol., 170:810 (1988) (nucleotide sequence of Streptococcus mutant fructosyltransferase gene); Steinmetz et al., Mol. Gen. Genet., 20:220 (1985) (nucleotide sequence of Bacillus subtilis levansucrase gene); Pen et al., Bio/Technology, 10:292 (1992) (production of transgenic plants that express Bacillus licheniformis amylase); Elliot et al., Plant Molec. Biol., 21:515 (1993) (nucleotide sequences of tomato invertase genes); Søgaard et al., J. Biol. Chem., 268:22480 (1993) (site-directed mutagenesis of barley amylase gene); and Fisher et al., Plant Physiol., 102:1045 (1993) (maize endosperm starch branching enzyme 11).

### Methods for Rice Transformation

Numerous methods for plant transformation are known in the art, including both biological and physical transformation protocols. See, e.g., Miki, et al., “Procedures for Introducing Foreign DNA into Plants” in Methods in Plant Molecular Biology and Biotechnology; Glick B. R. and Thompson, J. E. (Eds.) (CRC Press, Inc., Boca Raton, 1993), pp. 67-88. In addition, expression vectors and in vitro culture methods for plant cell or tissue transformation and regeneration of plants are known in the art. See, e.g., Gruber et al., “Vectors for Plant Transformation” in Methods in Plant Molecular Biology and Biotechnology, Glick B. R. and Thompson, J. E. (Eds.) (CRC Press, Inc., Boca Raton, 1993), pp. 89-119.

### A. Agrobacterium-Mediated Transformation

One method for introducing an expression vector into plants is based on the natural transformation system of Agrobacterium. See, e.g., Horsch et al., Science, 227:1229 (1985). A. tumefaciens and A. rhizogenes are plant pathogenic soil bacteria that genetically transform plant cells. The Ti and Ri plasmids of A. tumefaciens and A. rhizogenes, respectively, carry genes responsible for genetic transformation of plants. See, e.g., Kado, C. I., Crit. Rev. Plant Sci., 10:1 (1991). Descriptions of Agrobacterium vector systems and methods for Agrobacterium-mediated gene transfer are provided by Gruber et al., supra; Miki et al., supra; and Moloney, et al., Plant Cell Reports, 8:238 (1989). See also U.S. Pat. No. 5,591,616.

### B. Direct Gene Transfer

Despite the fact the host range for Agrobacterium-mediated transformation is broad, it is more difficult to transform some cereal crop species and gymnosperms via this mode of gene transfer, although success has been achieved in both rice and corn. See Hiei et al., The Plant Journal, 6:271-282 (1994); and U.S. Pat. No. 5,591,616. Other methods of plant transformation exist as alternatives to Agrobacterium-mediated transformation.

A generally applicable method of plant transformation is microprojectile-mediated (so-called “gene gun”) transformation, in which DNA is carried on the surface of microprojectiles, typically 1 to 4 m in diameter. The expression vector is introduced into plant tissues with a biolistic device that accelerates the microprojectiles to typical speeds of 300 to 600 m/s, sufficient to penetrate plant cell walls and membranes. Sanford et al., Part. Sci. Technol., 5:27 (1987); Sanford, J. C., Trends Biotech., 6:299 (1988); Klein et al., Bio/Technology, 6:559-563 (1988); Sanford, J. C., Physiol Plant, 7:206 (1990); and Klein et al., Biotechnology, 10:268 (1992). Various target tissues may be bombarded with DNA-coated microprojectiles to produce transgenic plants, including, for example, callus (Type I or Type II), immature embryos, and meristematic tissue.

Another method for physical delivery of DNA to plants is sonication of target cells. Zhang et al., Bio/Technology, 9:996 (1991). Alternatively, liposome or spheroplast fusion has been used to introduce expression vectors into plants. Deshayes et al., EMBO J., 4:2731 (1985); and Christou et al., Proc Natl. Acad. Sci. U.S.A., 84:3962 (1987). Direct uptake of DNA into protoplasts, using CaCl₂ precipitation, polyvinyl alcohol, or poly-L-ornithine, has also been reported. Hain et al., Mol. Gen. Genet., 199:161 (1985); and Draper et al., Plant Cell Physiol., 23:451 (1982). Electroporation of protoplasts and whole cells and tissues has also been described. Donn et al., in Abstracts of VIIth International Congress on Plant Cell and Tissue Culture IAPTC, A2-38, p. 53 (1990); D'Halluin et al., Plant Cell, 4:1495-1505 (1992); and Spencer et al., Plant Mol. Biol., 24:51-61 (1994).

Following transformation of rice target tissues, expression of a selectable marker gene allows preferential selection of transformed cells, tissues, or plants, using regeneration and selection methods known in the art.

These methods of transformation may be used for producing a transgenic inbred line. The transgenic inbred line may then be crossed with another inbred line (itself either transformed or non-transformed), to produce a new transgenic inbred line. Alternatively, a genetic trait that has been engineered into a particular rice line may be moved into another line using traditional crossing and backcrossing techniques. For example, backcrossing may be used to move an engineered trait from a public, non-elite inbred line into an elite inbred line, or from an inbred line containing a foreign gene in its genome into an inbred line or lines that do not contain that gene.

The term “inbred rice plant” should be understood also to include single gene conversions of an inbred line. Backcrossing methods can be used with the present invention to improve or introduce a characteristic into an inbred line.

Many single gene traits have been identified that are not regularly selected for in the development of a new inbred line, but that may be improved by crossing and backcrossing. Single gene traits may or may not be transgenic. Examples of such traits include male sterility, waxy starch, herbicide resistance, resistance for bacterial or fungal or viral disease, insect resistance, male fertility, enhanced nutritional quality, yield stability, and yield enhancement. These genes are generally inherited through the nucleus. Known exceptions to the nuclear genes include some genes for male sterility that are inherited cytoplasmically, but that still act functionally as single gene traits. Several single gene traits are described in U.S. Pat. Nos. 5,777,196; 5,948,957; and 5,969,212.

### Deposit Information

Samples of each of the rice lines 16HT195, 16HT489, 16HT498, and 16HT695 were deposited with the Provasoli-Guillard National Center for Marine Algae and Microbiota, Bigelow Laboratory for Ocean Science, 60 Bigelow Drive, East Boothbay, Maine 04544, United States (NCMA) on 9 Apr. 2021; and were assigned NCMA Accession Nos. 202104013, 202104010, 202104012, and 202104011, respectively. Each of these four, separate, individual deposits was made under the Budapest Treaty.

### Miscellaneous

The complete disclosures of all references cited in this specification are hereby incorporated by reference. Also incorporated by reference is the complete disclosure of the priority application, U.S. provisional patent application 63/178,591, including all its Appendices. Also incorporated by reference is the complete disclosure of D. Rebong, Development of Provisia® Hybrids for the Louisiana Rice Industry, PhD Dissertation, Louisiana State University, Baton Rouge, Louisiana (May 2021). In the event of an otherwise irreconcilable conflict, however, the present specification shall control.

What is claimed:

1. A rice plant of an environment-sensitive genic male sterility (EGMS) line selected from the group consisting of 16HT195, 16HT489, 16HT498, and 16HT695; a representative sample of seeds of each of said rice lines having been deposited under NCMA Accession Nos. 202104013, 202104010, 202104012, and 202104011, respectively; or an F₁ hybrid of one of said EGMS rice lines.

2. The rice plant of claim 1, wherein said rice line is an F₁ hybrid of 16HT195 or an F₁ hybrid of 16HT695; wherein said F₁ hybrid expresses the ACCase herbicide resistance characteristics of 16HT195 or 16HT695.

3. Rice seed capable of producing the rice plant of claim 2.

4. The rice plant of claim 1, wherein said rice plant is an F₁ hybrid of 16HT489 or an F₁ hybrid of 16HT498.

5. Rice seed capable of producing the rice plant of claim 4.

6. Rice seed of the rice plant of claim 1, or rice seed capable of producing said rice plant.

7. The rice plant of claim 1, wherein said rice plant is 16HT195.

8. Rice seed of the rice plant of claim 7, or rice seed capable of producing said rice plant.

9. The rice plant of claim 1, wherein said rice plant is an F₁ hybrid of 16HT195.

10. Rice seed capable of producing the rice plant of claim 9.

11. The rice plant of claim 1, wherein said rice plant is 16HT489.

12. Rice seed of the rice plant of claim 11, or rice seed capable of producing said rice plant.

13. The rice plant of claim 1, wherein said rice plant is an F₁ hybrid of 16HT489.

14. Rice seed capable of producing the rice plant of claim 13.

15. The rice plant of claim 1, wherein said rice plant is 16HT498.

16. Rice seed of the rice plant of claim 15, or rice seed capable of producing said rice plant.

17. The rice plant of claim 1, wherein said rice plant is an F₁ hybrid of 16HT498.

18. Rice seed capable of producing the rice plant of claim 17.

19. The rice plant of claim 1, wherein said rice plant is 16HT695.

20. Rice seed of the rice plant of claim 19, or rice seed capable of producing said rice plant.

21. The rice plant of claim 1, wherein said rice plant is an F₁ hybrid of 16HT695.

22. Rice seed capable of producing the rice plant of claim 21.

23. The seed of claim 3, wherein said seed is treated with an ACCase-inhibiting herbicide.

24. The seed of claim 23, wherein the ACCase-inhibiting herbicide comprises an aryloxyphenoxy herbicide.

25. The seed of claim 23, wherein the ACCase-inhibiting herbicide comprises a cyclohexanedione herbicide.

26. Pollen of the plant of claim 1.

27. An ovule of the plant of claim 1.

28. A composition comprising a product prepared from the rice plant of claim 1.

29. A tissue culture of regenerable cells or protoplasts produced from the rice plant of claim 1.

30. The tissue culture of claim 29, wherein said regenerable cells or protoplasts are produced from a tissue selected from the group consisting of embryos, meristematic cells, pollen, leaves, anthers, roots, root tips, flowers, seeds, and stems.

31. A method for producing rice plants, said method comprising planting a plurality of rice seeds of the rice plant of claim 1, or a plurality of rice seeds capable of producing said rice plant, under conditions favorable for the growth of rice plants.

32. A method for producing rice plants, said method comprising planting a plurality of rice seeds capable of producing the rice plant of claim 2, under conditions favorable for the growth of rice plants; and additionally comprising the step of applying herbicide in the vicinity of the rice plants, wherein the herbicide normally inhibits acetyl-CoA carboxylase, at levels of the herbicide that would normally inhibit the growth of a rice plant.

33. The method of claim 32, further comprising applying the herbicide to weeds in the vicinity of the rice plants.

34. The method of claim 32, wherein the herbicide comprises an aryloxyphenoxy herbicide.

35. The method of claim 32, wherein the herbicide comprises a cyclohexanedione herbicide.

36. The method of claim 32, wherein the herbicide comprises at least one of quizalofop, quizalofop-P, quizalofop-P-ethyl, quizalofop-P-tefuryl, haloxyfop, haloxyfop-P, fluazifop, cycloxydim, sethoxydim, tepraloxydim, or mixtures thereof.

37. A method of producing a rice plant, said method comprising transforming the rice plant of claim 1 with a transgene that confers insect resistance; a transgene that confers disease resistance; or a transgene encoding a protein selected from the group consisting of fructosyltransferase, levansucrase, alpha-amylase, invertase, and starch-branching enzyme; or a transgene encoding an antisense sequence to stearyl-ACP desaturase.

38. A rice plant or rice seed produced by the method of claim 37.

39. A method of introducing a desired trait into an EGMS line selected from the group consisting of 16HT195, 16HT489, 16HT498, and 16HT695; a representative sample of seeds of each of these lines having been deposited under NCMA Accession Nos. 202104013, 202104010, 202104012, and 202104011; said method comprising the steps of:
(a) crossing plants as recited in claim 1 with plants of another rice line expressing the desired trait, to produce progeny plants;
(b) selecting progeny plants that express the desired trait, to produce selected progeny plants;
(c) crossing the selected progeny plants with plants as recited in claim 1 to produce new progeny plants;
(d) selecting new progeny plants that express both the desired trait and some or all of the physiological and morphological characteristics of the EGMS line, to produce new selected progeny plants; and
(e) repeating steps (c) and (d) three or more times in succession, to produce selected higher generation backcross progeny plants that express both the desired trait and essentially all of the physiological and morphological characteristics of the EGMS line, as described in the specification, determined at a 5% significance level, when grown in the same environmental conditions.

40. Rice seed from a progeny plant produced by the method of claim 39; wherein, if said rice seed is grown, then the rice plants grown from said rice seed will express the ACCase herbicide resistance characteristics of 16HT195 or 16HT695.

41. The method of claim 39, additionally comprising the step of planting a plurality of rice seed produced by selected higher generation backcross progeny plants under conditions favorable for the growth of rice plants.

42. The method of claim 41, additionally comprising the step of applying herbicide in the vicinity of the rice plants to control weeds, wherein the herbicide normally inhibits acetyl-CoA carboxylase, at levels of the herbicide that would normally inhibit the growth of a rice plant.

43. The method of claim 42, wherein the herbicide comprises an aryloxyphenoxy herbicide.

44. The method of claim 42, wherein the herbicide comprises a cyclohexanedione herbicide.

45. The method of claim 42, wherein the herbicide comprises at least one of quizalofop, quizalofop-P, quizalofop-P-ethyl, quizalofop-P-tefuryl, haloxyfop, haloxyfop-P, fluazifop, cycloxydim, sethoxydim, tepraloxydim, or mixtures thereof.

46. The method of claim 39, wherein the selected progeny plants are F₁ hybrid plants.
